(19) United States
(12) Patent Application Publication
(10) Pub. No.: US 2022/0088213 A1
(43) Pub. Date: Mar. 24, 2022

(54) DRUG DELIVERY SYSTEM USING PH-DEPENDENT CELL-PENETRATING PEPTIDES, AND COMPOSITE THEREOF WITH DRUG

(71) Applicant: Sung Chun KIM, Seoul (KR)

(72) Inventor: Sung Chun KIM, Seoul (KR)

(21) Appl. No.: 17/420,168

(22) PCT Filed: Jan. 3, 2020
(86) PCT No.: PCT/KR2020/000116

§ 371 Date: Jul. 1, 2021

(30) Foreign Application Priority Data
Jan. 3, 2019 (KR) 10-2019-0000836
Aug. 14, 2019 (KR) 10-2019-0099364

(51) Int. Cl. A61K 47/68; A61K 45/06; A61K 31/337

(52) U.S. Cl. CPC A61K 47/6851; A61K 31/337; A61K 47/6803; A61K 45/06

(57) ABSTRACT

The present invention provides a drug delivery system using a pH-dependent cell-penetrating peptide and to a composite thereof with a drug. The drug delivery system of the present invention selectively (or specifically) acts only on specific target cells, thereby reducing side effects of a drug and enhancing drug efficacy, and can be usefully used to deliver drugs such as anticancer agents, immunosuppressants, contrast agents, etc.

### TECHNICAL FIELD

The present invention relates to a drug delivery system using a pH-dependent cell-penetrating peptide and to a drug and drug delivery system conjugate including same.

### BACKGROUND ART

Interest in developing an effective drug delivery system for delivering various drugs (for example, small molecule cytotoxic anticancer drugs, recombinant proteins, genes, contrast agents, etc.) to specific organs, tissues, and cells is growing. Typically, these systems are achieved using substances that bind specifically and strongly to molecules that are specifically present in specific cells. Unlike traditional formulations, target-specific therapeutic agents have been designed to maximize the bioavailability of a therapeutic agent delivered to a target site and are known to increase a therapeutic effect while treating diseases with few side effects. This drug delivery technology is a high value-added technology and plays an increasingly important roll in the overall drug development process.

Recently, attempts have been made to use cell-penetrating peptides (CPPs), glycosylated triterpenoids, etc. to efficiently deliver various drugs (for example, DNA, siRNA, peptides, and proteins) into cells (Morris et al., Nat. Biotechnol. 19(2001) 1173-1176; Jarver et al., Drug Discov. Today 9(2004) 395-402; and Pharmaceuticals 2012, 5, 1177-1209). Drug delivery using cell-penetrating peptides (CPP) is drawing great attention because it can increase the efficiency of delivery of macromolecules such as therapeutic peptides, proteins, and genes which have been difficult to be used as drugs in the case of non-invasive drug administration.

On the other hand, nanoparticle drug delivery systems (NDDs) have been extensively studied over the past few decades and have attracted great attention in the development of cancer-targeted therapeutics. NDDs alter the biodistribution and pharmacokinetic properties of drugs to mitigate side effects and enhance therapeutic effects. These positive effects are attributable to specific binding to tumor or vascular cells, enhanced permeability and retention (EPR) effects, tumor intrinsic pathophysiology, and usability of microenvironment of NDDs (for example, nanoparticles sensitive to pH, redox, enzymes, or other stimuli).

The present invention discloses a drug delivery system capable of delivering a drug specifically to a particular target cell and discloses a complex thereof with a drug.

### SUMMARY

### Technical Problem

One objective of the present invention is to provide a drug delivery system capable of reducing side effects of drugs and enhancing efficacy of drugs by selectively (or specifically) acting only on target cells.

Another objective of the present invention is to provide a drug and the drug delivery system conjugate.

Other objectives and intentions will be understood from the following description.

### Technical Solution

In order to achieve one of the above objectives, the present invention provides a drug delivery system including a cell targeting domain to which a cell-penetrating peptide (CPP) is bound, the cell targeting domain being a domain that specifically recognizes and binds with a target molecule expressed on the surface of a specific target cell, the cell-penetrating peptide (CPP) being capable of increasing efficiency of drug release or drug delivery from the outside to the inside of a cell or from endosomes to cytoplasm in a cell.

To evaluate whether the drug delivery system configured as described above has the intended effects, i.e., the effect of acting selectively (or specifically) on specific target cells to reduce drug side effects and the effect of increasing drug release efficiency to enhance drug efficacy, the inventors prepared drug delivery systems as in examples described below in which the cell targeting domain is composed of an aptamer or antibody that specifically recognizes and binds to HER2 that is a target cell, and the cell-penetrating peptide is composed of a pH-independent cell-penetrating peptide selected from among Melittin, LP, and Hylin a1 each of which has pH-independent cell-penetrating activity, or a pH-dependent cell-penetrating peptide selected from among LPE3-1 and pHD24 each of which has pH-dependent cell-penetrating activity. In addition, the inventors prepared composites in each of which the drug delivery system is bound to an apoptotic drug such as PLK1 siRNA or paclitaxel, treated BT-474 cells that overexpress HER2 and MDA-MB-231 cells that do not express HER2 with the prepared composites under various pH conditions to evaluate the selective action of the drug and the degree of enhancement of the drug efficacy.

Here, the HER2 is a member of the human epidermal growth factor receptor (HER/EGFR/ErbB) family and is known as an important biomarker and a therapeutic target in breast cancer patients (Nature Clinical Practice Oncology, 2006, 3:269-280; World J Clin). Oncol. 2017, 8(2):120-134). The PLK1 is a regulator that plays a central role in cell division (Cell Rep. 2013, 3(6):2021-32) and is a factor that is an important target for anticancer treatment because it is overexpressed in various human tumor cells (Transl Oncol. 2017, 10(1):22-32). Paclitaxel is a diterpenoid anticancer drug widely used as an anticancer medication for breast cancer and uterine cancer.

According to the results of the evaluation, the drug delivery systems respectively using Melittin, LP, and Hylin a1, that are cell-penetrating peptides, induced an apoptosis effect by acting on both the BT-474 cells overexpressing the HER2 gene and MDA-MB-231 cells not expressing the HER2 gene at a pH of about 7.0 that is similar to the pH condition of major tissues of the body, such as the cytoplasm or blood and to the pH of the extracellular environment. Thus, these drug delivery systems did not show selective action depending on whether HER2 genes that were targets were expressed or not. However, the drug delivery systems respectively using OPE301 and pHD24, which are pH-dependent cell-penetrating peptides, selectively acted on BT-474 cells in which the HER2 gene is overexpressed but hardly acted on MDA-MB-231 cells in which the HER2 gene is not expressed. That is, these drug delivery systems induced an apoptosis effect on the BT-474 cells but did not induce an apoptosis effect on the MDA-MB-231 cells.

On the other hand, as control groups, a drug delivery system and a composite thereof were configured such that the drug delivery system includes a HER2-specific aptamer or antibody bound to the drug “PLK1 siRNA” or “paclitaxel” but does not include the pH-dependent cell-penetrating peptide “LPE3-1”. The control groups also selectively acted and induced an apoptotic effect on BT-474 cells overexpressing HER2. However, the apoptotic effect of the control groups was significantly lower than that of the drug delivery system including the pH-dependent cell-penetrating peptide “LPE3-1” or the drug composite thereof. On the other hand, as seen from the examples described below, the composite of the pH-dependent cell-penetrating peptide and the drug “PLK1 siRNA” or “paclitaxel” did not show a selective action of the drug at pH 7 and exhibited little apoptosis.

According to the results of the experiment, in the case of the drug delivery system composed of a cell targeting domain and a pH-independent cell-penetrating peptide, the peptide acts on a cell membrane at pH 7, thereby directly delivering a drug into the cell. On the other hand, in the case of the drug delivery system composed of a cell targeting domain and a pH-dependent cell-penetrating peptide, the peptide does not act on a cell membrane at pH 7, but the cell targeting domain binds to the target molecule of the target cell, internalizes into the endosome, and is activated in a low-pH (for example, about 4 to 6) endosome or lysosome to release the drug into the cytoplasm. As confirmed from the examples below, at pH 5.5, both the pH-dependent cell-penetrating peptide and the pH-independent cell-penetrating peptide exhibited a similar degree of apoptosis effect on BT-474 cells and MDA-MB-231 cells, i.e., regardless of the presence or absence of overexpression of the target molecule in the treated cells, when the peptides are used in the form of drug delivery systems, each including either one of the peptides and a cell targeting domain.

In one aspect, the present invention may be regarded as a drug delivery system in which a cell targeting domain and a pH-dependent cell-penetrating peptide are combined, and in another aspect, the drug delivery system may be regarded as a conjugate in which the drug delivery system is bound to a drug.

In the specification of the present patent application, the pH-dependent cell-penetrating peptide refers to a peptide that does not exhibit cell permeability at about pH 7 but exhibits cell permeability under acidic conditions, specifically, in a pH range of 4 to 6.5. In other words, it refers to a peptide that does not exhibit cell membrane permeation activity under an extracellular environmental condition of about pH 7 but exhibits cell membrane permeation activity in endosomes or lysosomes having acidic conditions.

Also, in the present invention, the target molecule of the target cell to which the cell targeting domain selectively recognizes and binds is any antigen or receptor present on the surface of a specific target cell.

The specific cell means any target cell for which a drug needs to be delivered into the cell. This target cell is usually a cancer cell (or cancer stem cell). The cancer cells mean all kinds of cancer cells and include, for example, cells of esophageal cancer, stomach cancer, colorectal cancer, rectal cancer, oral cancer, pharyngeal cancer, laryngeal cancer, lung cancer, colon cancer, breast cancer, cervical cancer, endometrial cancer, ovarian cancer, prostate cancer, testicular cancer, bladder cancer, kidney cancer, liver cancer, pancreatic cancer, bone cancer, connective tissue cancer, skin cancer, brain cancer, thyroid cancer, leukemia, Hodgkin's disease, lymphoma, multiple myeloma, and blood cancer. In addition to cancer cells, any abnormal cells that require drug delivery into the cells may also be target cells. Examples of such abnormal cells include enlarged prostate cells, thyroid cells with hyperimmune activity, and cells associated with an autoimmune disease (for example, B cells that produce antibodies associated with rheumatoid arthritis, lupus, myasthenia gravis, etc.). In addition, the target cell may be a normal cell. For example, any normal cells such as dendritic cells, endothelial cells of blood vessels, lung cells, breast cells, bone marrow cells, spleen cells, thymocytes, liver cells, ovarian cells, etc. may be the target cells. When these normal cells are used as target cells, they may be used as a control group for determining or confirming drug effects on cancer cells or abnormal cells. The target cell may be an in vivo cell constituting a living animal or human tissue, or an in vitro cell such as a cultured animal cell, a cultured human cell, or a microorganism.

Also, the target molecule to which the cell targeting domain selectively recognizes and binds is any antigen or receptor present on the surface of a spedfic target cell. The antigen preferably refers to an antigen overexpressed in target cells compared to non-target cells, particularly including any cell surface receptor overexpressed in cancer cells compared to normal cells. Example of the target molecule include epidermal growth factor receptors (EGFR) overexpressed in anaplastic thyroid cancer, breast cancer, lung cancer, etc., metastin receptors overexpressed in papillary thyroid cancer, ErbB receptor tyrosine kinases overexpressed in breast cancer, human epidermal growth factor receptor 2 (HER2) overexpressed in breast cancer, tyrosine kinase-18-receptor (c-Kit) overexpressed in nutmegous renal carcinoma, HGF receptor c-Met overexpressed in esophageal adenocarcinoma, CXCR4 or CCR7 overexpressed in breast cancer, endothelin-A receptor overexpressed in prostate cancer, peroxisome proliferator activated receptor δ (PPAR-δ) overexpressed in rectal cancer, and platelet-derived growth factor receptor α (PDGFR-α) overexpressed in ovarian cancer. In addition, CD44, CD133, CD166, etc., which are surface antigens of cancer stem cells, may be target molecules (Cancer Res, 2005, 65(23)10946-51; Cancer Res, 2007, 67(3): 1030-7). Aside from these, carcinoembryonic antigen (CEA), prostate spedfic membrane antigen (PSMA), tumor-associated glycoprotein 72 (TAG-72), GD2 ganglisoside, GD3 ganglisoside, human leukocyte antigen-DR (HLA-DR10), tumor-associated antigen L6 (TAL6), tumor-necrosis factor-related apoptosis-inducing ligand receptor (TRAILR2), vascular endothelial growth factor receptor 2 (VEGFR2), hepatocyte growth factor receptor (HGFR), etc. may also be target molecules.

In the present invention, the cell targeting domain provides a targeting function by enabling selective binding to a target cell. This cell targeting domain specifically binds to an antigen or receptor present on the surface of a target cell to induce endocytosis, thereby enabling intrusion of the drug bound thereto into the cell.

Examples of the cell targeting domain (CTD) include antibodies, aptamers, hormones (for example, erythropoietin hormone) that are secreted from a spedfic cell and acts on the surface receptor of another cell to perform signal transmission between cells, cytokines or chemokines (for example, IL13), ligands, which are biomolecules such as a vascular endothelial growth factor (VEGF) and a brain-derived neurotrophic factor (BDNF) that bind to target cell surface receptors, and peptides, which are part of these factors with spedfic binding ability to receptors.

Typically, the cell targeting domain in the drug delivery system of the present invention is an antibody or an aptamer.

Antibodies as cell targeting domains are monoclonal antibodies, polyclonal antibodies, as well as multispedfic antibodies (that is, antibodies that recognize two or more antigens or two or more epitopes and which refer to bispecific antibodies, etc.). Alternatively, the antibodies may be fragments of antibodies, chemically modified antibodies, and chimeric antibodies (human and mouse chimeric antibodies, human and monkey chimeric antibodies, etc.). The antibody refers to any antibody such as a humanized antibody with reduced immunogenicity or a human antibody as long as it has the ability to specifically bind to a target antigen. In addition, various forms of antibody fragments and chemically modified antibodies are known in the art. For example, examples thereof include Fab, F(ab′)2, scFv (antibodies in which Fv of heavy and light chains are linked with suitable linkers), Fv, Fab/c (antibody having one Fab and complete Fc), and antibody fragments obtained by treating an antibody with a proteolytic enzyme such as papain or pepsin.

As the antibody that can serve as a cell targeting domain, antibodies that have been developed and commercially available may be used. Examples thereof include Cetuximab, Trastuzumab, Oregovomab, Edrecolomab, Alemtuzumab, Labetuzumab, Bevacizumab, Ibritumomab, Ofatumumab, Panitumumab, Rituximab, Tositumomab, Ipilimumab, Gemtuzumab, Brentuximab, Vadastuximab, Glebatumumab, Depatuxizumab, Polatuzumab, and Denintuzumab.

Regarding an antibody production method and an antibody obtained by artificially modifying a natural antibody to improve the specificity for a target antigen or to increase immunogenicity, reference may be made to the following literatures: U.S. Pat. Nos. 4,444,887, 4,716,111, 5,545,806, and 5,814,318; International Patent Publication Nos. WO98/46645, WO98/50433, WO98/24893, WO98/16654, WO96/34096, and WO96/33735; Protein Eng 1994, 7(6):805-814; Proc Natl Acad Sci USA 1994, 91:969-973; and the like.

The aptamer as a cell targeting domain may be a single-stranded DNA aptamer or a single-stranded RNA aptamer. The aptamer refers to a nucleic acid ligand capable of specifically binding to a target molecule, such as a target antigen, like an antibody. It does not matter that the aptamer is a double-stranded DNA or RNA aptamer if it is possible to specifically bind to a target molecule. Methods of preparing and selecting aptamers capable of specific binding to a target molecule are all known in the art. Specifically, SELEX technique may be used as the aptamer preparation and selection method. This SELEX technique is the abbreviation of “Systematic Evolution of Ligands by Exponential Enrichment”. For the technique, reference may be made to the following literatures: Science 249 (4968):505-510, 1990; U.S. Pat. Nos. 5,475,096; 5,270,163; and International Patent Publication No. WO91/19813. Regarding a specific method for the selection of aptamers, or the use of appropriate reagents, materials, etc., reference may be made to the literatures [Methods Enzymol 267:275-301, 1996], [Methods Enzymol 318:193-214, 2000], and the like. The aptamer may be modified from sugar, phosphate and/or base to improve half-life in vivo. Nucleotides obtained by modifying sugars, phosphates, and/or bases, and preparation methods thereof are known in the art. For example, nucleotides obtained by modifying sugar include ones in which a hydroxyl group (OH group) of the sugar is modified with a halogen group, an aliphatic group, an ether group, an amine group, or the like. In addition, such nucleotides include ones in which ribose or deoxyribose that is a sugar itself is substituted with sugar analogs such as a-anomeric sugars. The sugar also may be substituted with epimeric sugars (for example, arabinose, xylose, and lyxoses), pyranose sugars, furanose sugars, or the like. The phosphate may be modified into P(O)S(thioate), P(S)S(dithioate), P(O)NR2(amidate), P(O)R, P(O)OR′, CO, or formacetal (CH₂). Herein, R or R′ is H or substituted or unsubstituted alkyl. When modified from phosphate, the linking group may be —O—, —N—, —S—, or —C—. Adjacent nucleotides bind to each other via this linking group.

In the drug delivery system of the present invention, the cell targeting domain is linked to a pH-dependent cell-penetrating peptide. This pH-dependent cell does not exhibit cell-penetrating activity on the cell membrane under the condition of about pH and is activated in an endosome or lysosome with a relatively low pH (for example, a pH range of 4 to 6) to exhibit transmembrane activity, thereby releasing drugs into the cytoplasm after the cell targeting domain binds to the target molecule of a target cell and is internalized into the cell as the endosome. Therefore, the drug delivery system of the present invention having a pH-dependent cell-penetrating peptide enables the drug efficacy to be selectively exhibited in target cells in which target molecules are expressed, without causing side effects that drug efficacy is non-selectively exhibited even in non-target cells that do not express target molecules due to the non-selective cell-penetrating activity of a pH-independent peptide of a drug delivery system.

Regarding the pH-dependent cell-penetrating peptide used in the drug delivery system of the present invention, several pH-dependent cell-penetrating peptides capable of binding to a cell targeting domain are known in the art. Examples of such peptides include: GALA peptide; pHD15, pHD24, and pHD108 peptides, which are variants of the MelP5 peptide; PE3-1, LPH4, and ATRAM peptides, which are variants of the LP peptide.

Regarding the GALA peptide, reference may be made to the literature [J. Am. Chem. Soc., 2015, 137:12199-12202, 2015]. For pHD15, pHD24, and pHD108, which are variants of the MelP5 peptide, reference may be made to the literature [J Am Chem Soc 2017, 139(2): 937-945]. For LPE3-1 and LPH4, which are variants of the LP peptide, reference may be made to the literature [Org Biomol Chem. 2016, 14(26):6281-8]. For the ATRAM peptide, reference may be made to the literature [Biochemistry 2015, 54:6567-6575]. All of these literatures are considered part of this specification as are all other literatures cited herein.

The literature [J. Am. Chem. Soc., 2015, 137:12199-12202, 2015] discloses that a GALA peptide that is derived from the N-terminal domain of the HA-2 subunit of influenza virus hemagglutinin and which consists of a repeating sequence of Glu-Ala-Leu-Ala, forms an α-helix structure and penetrates a cell membrane under acidic pH conditions but cannot penetrate the cell membrane at neutral pH.

The literature [J Am Chem Soc 2017, 139(2): 937-945] discloses that pHD15, pHD24, and pHD108, which are variants of the MelP5 peptide, exhibit high cell-penetrating activity under acidic conditions, that is, around pH 5 but do not exhibit cell-penetrating activity under neutral conditions close to pH 7. In addition, the literature [Org Biomol Chem. 2016, 14(26):6281-8] discloses that LPE3-1 and LPH4, which are variants of LP peptide, exhibit high cell-penetrating activity at round pH 5 but hardly exhibit cell-penetrating activity at pH 7.4.

The literature [Biochemistry 2015, 54:6567-6575] discloses that ATRAM peptide exhibits cell-penetrating activity only at a slightly acidic pH similar to that of the extracellular environment of solid tumors.

The amino acid sequences of the exemplified pH-dependent cell-penetrating peptides can be found below.

LPE3-1:
(SEQ ID NO: 1)
H2N-GWWLALAEAEAEALALASWIKRKRQQ-COOH
LPH4:
(SEQ ID NO: 2)
GWWLALALALALALALASWIHHHHQQ-COOH
pHD15:
(SEQ ID NO: 3)
H2H-GIGEVLHELADDLPDLQEWIHAAQQL-COOH
pHD24:
(SEQ ID NO: 4)
H2N-GIGDVLHELAADLPELQEWIHAAQQL-COOH
pHD108:
(SEQ ID NO: 5)
H2N-GIGEVLHELAEGLPELQEWIHAAQQL-COOH
ATRAM:
(SEQ ID NO: 6)
GLAGLAGLLGLEGLLGLPLGLLEGLWLGLELEGN-COOH

In the drug delivery system of the present invention, the cell-targeting domain and the pH-dependent cell-penetrating peptide may be directly covalently bound to each other without the mediation of a linker or may be covalently bound to each other via a linker.

When the cell-targeting domain is a protein such as an antibody, ligand, peptide, or cytokine, direct covalent binding to the pH-dependent cell-penetrating peptide is achieved by chemically joining the carboxyl group (or amino group) of the terminal amino acid of the cell-targeting domain which is a protein with the amino group (or carboxyl group) of the amino acid at the end of the pH-dependent peptide in a manner known in the art. In addition, such binding involves inserting a recombinant nucleic acid encoding these conjugates into an appropriate expression vector, and transforming the expression vector in an appropriate host microorganism (E. coli, HO cells, NSO cells, Sp2/0 cells, COS cells, animal cells such as HEK cells, etc.) so as to be expressed in the form of a fusion protein. The binding further involves isolation and purification. In the related art, recombinant nucleic acid technology, construction of expression vectors, selection marker genes, transformation methods, host microorganisms, composition of a culture medium for culturing host microorganisms, culturing methods, high-yield culturing methods, target-protein isolation methods, and the like are all known. Regarding these, a considerable amount of literature has been accumulated, and thus reference can be made thereto. For example, reference may be made to the literature [Sambrook et al., Molecular Cloning: A Laboratory Manual, 2nd Ed., Cold Spring Harbor Laboratory Press, Cold Spring Harbor, N.Y. (1989)], the literature [Sambrook et al., Molecular Cloning, A Laboratory Manual, Cold Spring Harbor Laboratory Press, (2001)], the literature [F M Ausubel et al, Current Protocols in Molecular Biology, John Wiley amp; Sons, Inc. (1994)], the literature [Marston, F (1987) DNA Cloning Techniques], etc.

When the cell targeting domain is a protein and forms a direct covalent bond with a pH-dependent peptide, several or tens of amino acids may be placed not to affect the specific binding property of the cell targeting domain to a target or not to affect the cell permeability of the pH-dependent peptide. The drug delivery system of the present invention in which such spacers are placed may be prepared by a chemical reaction or by the same recombinant fusion protein manufacturing method.

When the cell-targeting region is a protein such as an antibody, ligand, peptide, or cytokine, the protein may be bound to the pH-dependent cell non-covalently through electrostatic interactions such as hydrogen bonding, hydrophobic interaction, and the like. For example, when the cell targeting domain, which is a protein, has a negatively charged surface, it can bind to a cationic pH-dependent cell-penetrating peptide through an electrostatic interaction. Similarly, when the cell targeting domain, which is a protein, includes a hydrophobic region, it can bind to a hydrophobic pH-dependent cell-penetrating peptide through an electrostatic interaction.

Even when the cell targeting domain is an aptamer, it can bind non-covalently to the pH-dependent cell-penetrating peptide through an electrostatic interaction (charge interaction), a hydrophobic interaction, or the like, without the mediation of a linker. Since the aptamer, which is an nucleic acid, is negatively charged, it binds to, for example, a cationic pH-dependent peptide through an electrostatic interaction.

In the drug delivery system of the present invention, the cell targeting domain may covalently bind to a pH-dependent cell-penetrating peptide via a linker.

In the present invention, the linker may be an arbitrary linker having a functional group that can bind to an amine group, a carboxyl group, or a sulfhydryl group of a protein such as a peptide, ligand, antibody, or antibody fragment, or to a phosphate group or a hydroxyl group of a nucleic acid such as an aptamer.

The linker has a functional group selected from among isothiocyanate, isocyanates, acyl azide, NHS ester, sulfonyl chloride, aldehyde, glyoxal, epoxide, oxirane, carbonate, aryl halide, imidoester, carbodiimide, anhydride, fluorophenyl ester, hydroxymethyl phosphine, maleimide, haloacetyl, pyridyldisulfide, thiosulfonate, and vinylsulfone.

The linker may be a linker cleavable by a protease, cleavable under acid or base conditions, cleavable under high temperature or light irradiation, or cleavable under reducing or oxidizing conditions, or may be a linker that is not cleavable under these conditions.

Examples of the cleavable linker include a hydrazone linker cleaved under acidic conditions, a peptide linker cleaved by a protease, and a linker having a disulfide functional group that is cleaved under reducing conditions. Examples of non-cleavable linkers include: a maleimidomethyl cyclohexane-1-carboxylate (MCC) linker, a maleimidocaproyl (MC) linker, and derivatives thereof, such as a succinimidyl-4-(N-maleimidomethyl)cyclohexane-1-carboxylate (sMCC) linker or a sulfosuccine imidyl-4-(N-maleimidomethyl)cyclohexane-1-carboxylate (sulfa-sMCC) linker.

The linker may be a self-immolative linker or a traceless linker that does not leave the trace thereof after cleavage. Examples of the self-immolative linker include a linker disclosed in U.S. Pat. No. 9,089,614 entitled “Hydrophilic Self-Immolative Linkers and Conjugates thereof”, and a linker disclosed in International Patent Publication No. WO2015/038426 titled “Self-Immolative Linkers Containing Mandelic Acid Derivatives, Drug-Ligand Conjugates For Targeted Therapies”. Examples of the traceless linker include a phenylhydrazide linker, an aryl-triazene linker, and a linker disclosed in the literature [Blaney, et al., “Traceless solid-phase organic synthesis,” Chem Rev. 102: 2607-2024 (2002)]

The linker may also be a homobifunctional linker (which is a linker having two or more identical reactive functional groups) or a heterobifunctional linker (which is a linker having two or more different reactive functional groups).

Examples of the homobifunctional linker include 3′3′-dithiobis(sulfosuccinimidyl propionate (DTSSP), disuccinimidyl suberate (DSS), bis(sulfosuccinimidyl)suberate (BS), disuccinimidyl tartrate (DST), disulfosuccinimidyl tartrate (Sulfa DST), ethylene glycobis(succinimidyl succinate)(EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido butane (DPDPB), bis-[β(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), N,N′-hexamethylene-bis(iodoacetam ide), etc.

Heterobiflunctional linkers include amine-reactive and sulfhydryl-reactive cross-linkers, carbonyl-reactive and sulfhydryl-reactive cross-linkers, amine-reactive and photoreactive cross-linkers, sulfhydryl-reactive and photoreactive cross-linkers, and the like. Examples of the amine-reactive and sulfhydryl-reactive cross-linkers include N-succinimidyl 3-(2-pyridyldithio)propionate (sPDP), long chain N-succinim idyl 3-(2-pyridyldithio) propionate (LC-sPDP), water-soluble-long-chain N-succinimidyl 3-(2-pyridyldithio) propionate (sulfo-LC-sP DP), succinimidyloxycarbonyl-α-methyl-α-(2-pyridyldithio)toluene (sMPT), sulfosuccinim idyl-6-[α-methyl-α-(2-pyridyldithio)toluamido]hexanoate (sulfo-LC-sMPT), succinim idyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfa-MBs), N-succinimidyl (4-iodoacetyl) aminobenzoate (sIAB), etc. Examples of the carbonyl-reactive and sulfhydryl-reactive crosslinkers include 4-(4-N-maleimidophenyl)butyric acid hydrazide (MPBH), 4-(N-maleimidomethyl)cyclohexane-1-carboxyl-hydra Zide-8 (M2C2H), 3-(2-pyridyldithio)propionyl hydrazide (PDPH), etc. Examples of the amine-reactive and photoreactive cross-linkers include N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2) nitrophenylamino)hexanoate (sulfo-sANPAH), N-5-azido-2-nitrobenzoyloxysuccinimide (ANB-NOs), and the like. Examples of the sulfhydryl-reactive and photoreactive cross-linker include 1-(p-azidosalicylamido)-4-(iodoacetamido)butane (AsIB), N-[4-(p-azidosalicylamido) and do)butyl]-3′-(2′-pyridyldithio)propionamide (APDP), benzophenone-4-iodoacetamide, and benzophenone-4-maleimide.

In some embodiments, the linker is a dendritic-type linker. The dendritic-type linker has a branched, multifunctional linker. Examples of such a linker include PAMAM dendrimers.

Aside from the linkers mentioned above, numerous linkers applicable to the present invention are known in the art and are disclosed in a considerable number of literatures. For example, as to the linkers, reference may be made to non-patent literatures including the literature [Castaneda, et al, “Acid-cleavable thiomaleamic acid linker for homogeneous antibodydrug conjugation,” Chem Commun. 49: 8187-8189 (2013)], the literature [Lyon, et al, “Self-hydrolyzing maleimides improve the stability and pharmacological properties of antibody-drug conjugates,” Nat Biotechnol. 32(10):1059-1062 (2014)], the literature [Dawson, et al “Synthesis of proteins by native chemical ligation,” Science 1994, 266, 776-779], the literature [Dawson, et al “Modulation of Reactivity in Native Chemical Ligation through the Use of Thiol Additives,” J Am Chem Soc. 1997, 119, 4325-4329], the literature [Hackeng, et al “Protein synthesis by native chemical ligation: Expanded scope by using straightforward methodology,” Proc Natl Acad Sci USA 1999, 96, 10068-10073], the literature [Wu, et al “Building complex glycopeptides: Development of a cysteine free native chemical ligation protocol,” Angew Chem Int Ed 2006, 45, 4116-4125], the literature [Geiser et al “Automation of solid-phase peptide synthesis” in Macromolecular Sequencing and Synthesis, Alan R Liss, Inc, 1988, pp 199-218], and the literature [Fields, G and Noble, R (1990) “Solid phase peptide synthesis utilizing 9-fluoroenylmethoxycarbonyl amino acids”, Int J Peptide Protein Res 35:161-214]. In addition, reference may be made to patent literatures including U.S. Pat. Nos. 6,884,869, 7,498,298, 8,288,352, 8,609,105, 8,697,688, U.S. Patent Application Publication No. 2014/0127239, U.S. Patent Application Publication No. 2013/028919, U.S. Patent Application Publication No. 2014/286970, U.S. Patent Application Publication No. 2013/0309256, U.S. Patent Application Publication No. 2015/037360, U.S. Patent Application Publication No. 2014/0294851, International Patent Application Publication No. WO2015/057699, International Patent Application Publication No. WO2014/080251, International Patent Application Publication No. WO2014/197854, International Patent Application Publication No. WO2014/145090, and International Patent Application Publication No. WO2014/177042.

In another embodiment of the drug delivery system of the present invention, the cell targeting domain and the pH-dependent cell-penetrating peptide are bound to each other via a biocompatible polymer serving as a mediator or a carrier.

The biocompatible polymer refers to a polymer having tissue compatibility and blood compatibility that do not cause tissue necrosis or blood coagulation when it comes into contact with living tissue or blood.

Preferably, the biocompatible polymer serving as a carrier suitable for the present invention is a synthetic polymer or a natural polymer.

According to a preferred embodiment of the present invention, the synthetic polymer as the biocompatible polymer is polyester, polyhydroxyalkanoates (PHAs), poly(α-hydroxyacid), poly(β-hydroxyacid), poly(3-hydroxybutyrate-co-valerate; PHBV), poly (3-hydroxypropionate) (PHP), poly(3-hydroxyhexanoate) (PHH), poly(4-hydroxyacid), poly(4-hydroxybutyrate), poly(4-hydroxy hydroxyvalerate), poly(4-hydroxyhexanoate), poly(esteramide), polycaprolactone, polylactide, polyglycolide, poly(lactide-co-glycolide) (PLGA), polydioxanon, polyorthoester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHA-PEG, ethylene vinyl alcohol copolymer (EVOH), poly urethanes, silicones, polyesters, polyolefins, polyisobutylene and ethylene-alphaolefin copolymers, styrene-isobutylene-styrene triblock copolymers, acrylic polymers and copolymers, vinyl halide polymers and copolymers, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketone, polyvinyl aromatics, Polystyrene, polyvinyl ester, polyvinyl acetate, ethylene-methyl methacrylate copolymer, acrylonitrile-styrene copolymer, ABS resin and ethylene-vinyl acetate copolymer, polyamide, alkyd resin, polyoxymethylene, polyimide, polyether, polyacrylate, polymethacrylate, polyacrylic acid-co-maleic acid, or polyaminoamine

Preferably, the natural polymer as the biocompatible polymer is chitosan, dextran, cellulose, heparin, hyaluronic acid, alginate, inulin, starch, or glycogen. Preferably, the biocompatible polymer suitable for the present invention is a polymer having a dendrimer structure. For example, a dendrimer of polyaminoamine may be used as the biocompatible polymer in the present invention.

Regarding the use of a biocompatible polymer as a carrier for a drug or the like as in the present invention, a considerable number of literatures are known in the art. Thus, for more specific information, reference may be made to literatures. The literatures include [Kiran Dhaliwal, “Biodegradable Polymers and their Role in Drug Delivery Systems” Biomedical Journal of Scientific & Technical Research, 2018, 11(1):8315-8320], [Avnesh Kumari, et al., “Biodegradable polymeric nanoparticles based drug delivery systems” Colloids and Surfaces B: Biointerfaces, 2010, 75(1):1-18], and [Kumaresh S Soppimath, et al., “Biodegradable polymeric nanoparticles as drug delivery devices” Colloids and Surfaces B: Biointerfaces, 2001, 70(1):1-20]

In another aspect, the present invention relates to a drug and drug delivery system conjugate in which the drug delivery system described above and a drug are combined. In the drug and drug delivery system conjugate of the present invention, the drug may be covalently bound to the cell-targeting domain or the pH-dependent cell-penetrating peptide of the drug delivery system via a linker or may be non-covalently bound without a linker.

As shown in FIG. 1, the drug and drug delivery system conjugate of the present invention is linked in the specific order of the drug, the cell targeting domain, and the pH-dependent cell-penetrating peptide, or in the specific order of the cell targeting domain, the drug, and the pH-dependent cell-penetrating peptide, or in the specific order of the cell targeting domain, the pH-dependent cell-penetrating peptide, and the drug. Alternatively, as shown in FIG. 1, a drug, the cell targeting domain, and the pH-dependent cell-penetrating peptide may be bound in an arbitrary order via a biocompatible polymer.

In the drug and drug delivery system conjugate of the present invention, as a linker for binding the drug to the drug delivery system, an appropriate linker may be selected depending on the drug from among the linkers exemplified in relation to the drug delivery system of the present invention. For example, a linker having an aldehyde reactive group may bound to a drug, and the resulting conjugate may be bound to the N-terminal amino group of an antibody (which is a cell targeting domain) of a drug delivery system.

The linker used for binding the drug delivery system to the drug is preferably a linker that is not cleaved because it is stable outside a target cell and is not cleaved even in endosomes or lysosomes which are under acidic conditions in a target cell. Since this linker is stable outside the target cell and is not cleaved, the drug can move into the target cell. In addition, since the linker is not cleaved in endosomes or rhizosomes which are under acidic conditions, the drug can move into the cytoplasm from the endosomes or rhizosomes.

In the drug and drug delivery system conjugate of the present invention, the drug may be non-covalently bound to the drug delivery system. For example, intercalator agents such as doxorubicin, which is a type of anticancer agent that exhibits an effect by intercalation with a nucleic acid, may be non-covalently bound to an aptamer in an intercalation manner when the aptamer is used as the cell targeting domain of the drug delivery system. Since the aptamer is an oligonucleotide molecule, nucleotide bases are stacked, and a drug can be coupled in an intercalation manner between the base stacks.

In the drug and drug delivery system conjugate of the present invention, the drug is not particularly limited as long as it is a drug that can move into cells and exert an effect in the cells. Examples of the drug include drugs composed of low molecular weight compounds, such as cytotoxic anticancer agents, or biopharmaceuticals such as recombinant proteins or siRNA. In addition, in terms of efficacy, examples of the drug include anti-inflammatory, analgesic, anti-arthritic, antispasmodic, anti-depressant, anti-psychotic, tranquilizer, anti-anxiety, narcotic, anti-Parkin's disease drugs, cholinergic agonists, anti-cancer agents, angiogenesis inhibitors, immunosuppressants, immunostimulants, antiviral, antibiotic, appetite suppressant, analgesic, anticholinergic, antihistamine, anti-migraine, hormone, coronary, vasodilator, contraceptive, antithrombotic, diuretic, antihypertensive, cardiovascular disease treatment, contrast agent, etc.

In the present invention, the drug is preferably a cytotoxic anticancer agent. Examples of the cytotoxic anticancer agent include antimetabolites, microtubulin targeting agents (tubulin polymerase inhibitor and tubulin depolymerization), alkylating agents, antimitotic agents, DNA cleavage agents, DNA cross-linker agents, DNA intercalator agents, and DNA topoisomerase inhibitors. As the metabolites, folic acid derivatives such as methotrexate, purine derivatives such as cladribine, pyrimidine derivatives such as azacytidine, doxyfluoridine, fluorouracil, etc. are known. As the microtubuline targeting agents, monomethyl auristatin E (MMAE), monomethyl auristatin F (MMAF), auristatin-based drugs such as dolastatin, maytansines, etc. are known in the art. Known examples of the alkylating agent include alkyl sulfonate preparations such as busulfan and treosulfan, nitrogen mustard derivatives such as bendamustine, cisplatin, heptaplatin, and platinum formulations such as heptaplatin. In addition, as the antimitotic agents, taxane preparations such as docetaxel and paclitaxel, vinca alkalids such as vinflunine, and podophyllotoxin derivatives such as etoposide, and the like are known in the art. As the DNA cleavage agent, calicheamicins are known in the art. As the DNK cross-liner agent, PBD duplexes and the like are known. In addition, as the DNA intercalator agent, doxorubicin and the like are known in the art. As the DNA topoisomerase inhibitor, SN-28 and the like are known in the art.

Examples of the drug include a gene, plasmid DNA, antisense oligonucleotide, siRNA, peptide, ribozyme, viral particle, immunomodulator, protein, contrast agent, and the like. More specifically, the drug may be a gene encoding Rb94, which is a mutant of a retinoblastoma tumor suppressor gene, or a gene encoding apoptin, which induces apoptosis only in tumor cells. Alternatively, the drug may be an antisense oligonucleotide (Sequence: 5′-TCC ATG GTG CTC ACT-3′) against HER-2 which is a therapeutic target, or a diagnostic contrast agent such as a Gd-DTPA material used as an MRI contrast agent.

The drug and drug delivery system conjugate of the present invention includes a pharmaceutically acceptable carrier and may be prepared as a pharmaceutical composition for oral or parenteral administration according to a conventional method known in the art. As used herein, “pharmaceutically acceptable carrier” refers to a carrier or diluent that does not irritate the organism and does not interfere with the biological activity and properties of the administered compound. Acceptable pharmaceutical carriers for compositions formulated as liquid solutions need to be sterile and biocompatible. At least one component selected from among saline, sterile water, Ringer's solution, buffered saline, albumin injection, dextrose solution, maltodextrin solution, glycerol, and ethanol may be used solely or in combination. Other conventional additives such as antioxidants, buffers, and bacteriostats may be added thereto as needed.

The carrier is not particularly limited, but in the case of oral administration, a binder, a lubricant, a disintegrant, an excipient, a solubilizer, a dispersing agent, a stabilizer, a suspending agent, a dye, a flavoring agent, etc. may be used in combination therewith. Alternatively, in the case of an injection, a buffer, a preservative, an analgesic agent, a solubilizer, an isotonic agent, a stabilizer, etc. may be used in combination therewith. In the case of topical administration, a base, excipient, lubricant, preservative, etc. may be used in combination therewith.

The formulation of the composition of the present invention can be prepared in various ways by mixing the composition of the present invention with a pharmaceutically acceptable carrier described above. For example, in the case of oral administration, the composition may be formulated into tablets, troches, capsules, elixirs, suspensions, syrups, wafers, and the like. In the case of injection, the composition may be prepared in the form of single-dose ampoules or multiple-dose ampoules. Alternatively, the composition may be formulated as a solution, suspension, tablet, pill, capsule, sustained release formulation, and the like.

Examples of the carrier, excipient, and diluent suitable for formulation include lactose, dextrose, sucrose, sorbitol, mannitol, xylitol, erythritol, maltitol, starch, acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. In addition, a filler, an anti-agglomeration agent, a lubricant, a wetting agent, a flavoring agent, a preservative, and the like may be additionally included.

In addition, the pharmaceutical composition of the present invention may be prepared by a conventional method and formulated into tablets, pills, powders, granules, capsules, suspensions, mixtures for internal use, emulsions, syrups, sterilized aqueous solutions, non-aqueous preparations, suspensions, emulsions, freeze-drying preparations, or suppositories.

In addition, the composition may be formulated, by a conventional method used in the pharmaceutical field, into a unit dosage form suitable for administration to the body of a patient. Preferably, the composition may be formulated into a useful formulation suitable for administration of peptide pharmaceuticals and may be administered by a commonly used manner in the art. For example, the composition may be orally or parentally administered. When parentally administered, dermal, intravenous, intramuscular, intraarterial, intramedullary, intrathecal, intraventricular, pulmonary, transdermal, subcutaneous, intraperitoneal, intranasal, gastrointestinal, topical, sublingual, vaginal, or rectal administration may be possible.

In addition, the conjugate may be used in combination with various pharmaceutically acceptable carriers such as physiological saline or organic solvents. In addition, carbohydrates such as glucose, sucrose, or dextran, antioxidants such as ascorbic acid or glutathione, chelating agents, low molecular weight proteins, or other stabilizers may be added to increase stability or absorbency of drugs.

Formulation of pharmaceutical compositions is known in the art, and specifically, reference may be made to the literature [Remington's Pharmaceutical Sciences (19th ed., 1995)] and the like. This literature is considered a part of this specification.

A preferred dosage of the pharmaceutical composition of the present invention is in a range of 0.001 mg/kg to 10 g/kg per day, preferably 0.001 mg/kg to 1 g/kg per day, depending on the patient's condition, weight, sex, age, severity of the disease, and the route of administration. Administration may be performed once or several times a day. Such dosages should not be construed as limiting the scope of the invention in any respect.

### Advantageous Effects

As described above, according to the present invention, it is possible to provide a drug delivery system capable of reducing drug side effects and increasing drug efficacy by selectively (or specifically) acting only on specific target cells, and a conjugate of a drug and the drug delivery system. The drug delivery system of the present invention can be usefully used as a drug carrier for anticancer agents, immunosuppressants, contrast agents, and the like.

### BRIEF DESCRIPTION OF THE DRAWINGS

FIG. 1 schematically shows the configuration of a conjugate of a drug delivery system and a drug, the drug delivery system being prepared by the present invention, in which CTD represents a cell targeting domain, DRD represents a cell-penetrating peptide and is a drug releasing domain, and Drug represents a drug;

FIG. 2 is an HPLC analysis result and a polyacrylamide gel electrophoresis (PAGE) image for a case where LPE3-1 peptide is used;

FIG. 3 is polyacrylamide gel electrophoresis (PAGE) images of a HER2 Ap/PLK1 siRNA SS conjugate, a PLK1 siRNA AS/peptide conjugate, and a conjugate of the former two conjugates;

FIG. 4 is an image when BT-474 cells overexpressing HER2 and MDA-MB-231 cells not expressing HER2 are treated with a HER2 Ap/PLK1 siRNA/LPE3-1 conjugate that is a conjugate of a drug and a drug delivery system;

FIG. 5 is an image showing the results of investigation of apoptosis when BT-474 cells overexpressing HER2 and MDA-MB-231 cells not expressing HER2 are treated with each of several drug and drug delivery system conjugates in which five drug delivery systems are used;

FIGS. 6 and 7 show apoptosis-inducing effects according to the treatment time (FIG. 6) and the treatment concentration (FIG. 7) when BT-474 cells overexpressing HER2 and MDA-MB-231 cells not expressing HER2 are treated with each of conjugates, each including a drug delivery system such as HER2 Ap, PLK1 siRNA, and LPE3-1 and a drug;

FIGS. 8 and 9 show apoptosis degrees obtained by measuring changes in mitochondrial membrane potential when BT-474 cells overexpressing HER2 and MDA-MB-231 cells not expressing HER2 are treated with each of five conjugates composed of a drug and respective drug delivery systems, under conditions of pH 7.0 (FIG. 8) and pH 5.5 (FIG. 9);

FIG. 10 is a result showing the degree of apoptosis when BT-474 cells overexpressing HER2 and MDA-MB-231 cells not expressing HER2 are treated with various drug delivery systems in a control group;

FIGS. 11 and 12 are results showing the degree of apoptosis according to treatment time when BT-474 cells overexpressing the HER2 gene are treated with the drug delivery systems in a test group; and

FIGS. 13 and 14 are results showing the degree of apoptosis according to treatment concentration when the BT-474 cells overexpressing the HER2 gene are treated with the drug delivery systems in a test group.

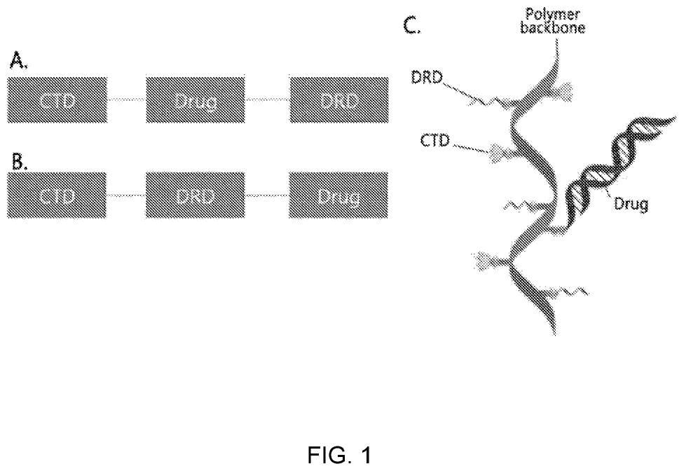

### DETAILED DESCRIPTION

Hereinafter, the present invention will be described with reference to various examples. However, the scope of the present invention is not limited by the examples.

### Example 1

### Preparation of Drug Delivery System for Selection of Peptide with Excellent Drug Release Efficiency

### Example 1-1

### Preparation of Raw Material of Drug Delivery System

A drug delivery system prepared in this example has CTD/drug/DRD structure in which a cell targeting domain (CTD), a drug, and a drug release domain (DRD) which is a drug release-inducing peptide are combined.

As the CTD, a human epidermal growth factor receptor 2-specific aptamer (HER2_Ap) was used. As the drug, siRNA that is specific for polo-like kinase 1 (PLK1) was used. As the DRD, five peptides listed in Table 1 below were used.

TABLE 1
Peptide type and amino acid sequence

| | | Sequence | |
|---|---|---|---|
| Type | Amino acid sequence | number | Note |
| Melittin | GIGAVLKVLTTGLPALISWIKRKRQQ | 7 | Activated over a |
| LP | GWWLALALALALALALASWIKRKRQQ | 8 | wide pH range |
| Hylin a1 | IFGAILPLALGALKNLIK | 9 | |
| LPE3-1 | GWWLALAEAEAEALALASWIKRKRQQ | 1 | Activated within an |
| pHD24 | GIGAVLKVLATGLPALISWIKAAQQL | 4 | acidic pH range |

Here, the HER2 is a member of the human epidermal growth factor receptor (HER/EGFR/ErbB) family and is known as an important biomarker and a therapeutic target in breast cancer patients (Nature Clinical Practice Oncology, 2006, 3:269-280; World J Clin Oncol. 2017, 8(2):120-134). In this example, a HER2-specific aptamer (Varmira K. et al., Nucl Med Biol. 2013, 40(8):980-6) the target molecule of which is HER2 was used as the CTD. The PLK1 is a regulator that plays a key role in cell division (Cell Rep. 2013, 3(6):2021-32) and is an important target for anticancer therapy because it is overexpressed in various human tumor cells (Transl Oncol. 2017, 10(1):22-32). In this example, PLK1 siRNA (Song WJ. et al., Small, 2010, 6(2):239-246) targeting PLK1 was used as a drug.

In order to prepare the drug delivery system of the present example, first, the single-stranded nucleic acid DNA “HER2 Ap/PLK1 siRNA SS” (SEQ ID NO: 10) having a HER2-specific aptamer (HER2 Ap) sequence, a spacer sequence (which is an underlined base sequence in SEQ ID NO: 10 shown below), and a sequence indicating the sense strand of PLK1 siRNA was obtained, by custom order, from Bioneer Corporation (in Korea). In addition, the single-stranded nucleic acid DNA “PLK1 siRNA SS” (SEQ ID NO: 11) indicating the sense strand of PLK1 siRNA was obtained, by custom order, from Bioneer corporation (in Korea). In addition, a single-stranded DNA (SEQ ID: 12) indicating the antisense strand “PLK1 siRNA AS” of PLK1 siRNA and peptides in Table 1 were obtained from BioSynthesis Incorporation (BSI in USA) by custom order.

HER2 Ap-spacer-PLK1 siRNA SS:
(SEQ ID NO: 10)
AGC CGC GAG GGG AGG GAA GGG TAG GGC
GCG GCT-TTTT-TGA AGA AGA TCA CCC TCC TTA TT
PLK1_siRNA_SS:
(SEQ ID NO: 11)
TGA AGA AGA TCA CCC TCC TTA TT
PLK1_siRNA_AS:
(SEQ ID NO: 12)
TAA GGA GGG TGA TCT TTC TTC A

### <Example 1-2

### Preparation of Conjugate of HER2-Specific Aptamer and PLK1 siRNA Sense Strand

For the production of a HER2 Ap/PLK1 siRNA SS conjugate, which is a conjugate of the HER2-specific aptamer and the PLK1 siRNA sense strand, first, with the use of the custom-made single-stranded nucleic acid of SEQ ID NO: 10 as a template, a PCR product was obtained by performing PCR using the forward primer of SEQ ID NO: 13 having a T7 promoter sequence (underlined base sequence in SEQ ID NO: 13 below) and the reverse primer of SEQ ID NO: 14 below. Next, using the PCR product as a template and using a 2′-F-substituted pyrimidine to perform in vitro transcription with the DuraScribe® T7 Transcription Kit (Lucigen, USA), a HER2 Ap-spacer-PLK1 siRNA SS conjugate was prepared. The conjugate is an RNA transcript containing a 2′-F-substituted pyrimidine and has higher in vivo stability than native RNA.

Forward primer with T7 promoter:
(SEQ ID NO: 13)
TAA TAC GAC TCA CTA TAG GGA GA AGC CGC
GAG GGG AGG GAA
Reverse Primer:
(SEQ ID NO: 14)
AAT AG GAG GGT GAT CTT

The PCR was performed using 1,000 pmoles of single-stranded nucleic acid DNA (SEQ ID NO: 10), 2,500 pmoles of PCR primer pairs, 50 mM of KCI, 10 mM of tris-CI (pH 8.3), 3 mM of MgCl₂, 0.5 mM of dNTP (dATP, dCTP, dGTP, and dTTP), and 0.1 U of Taq DNA polymerase (manufactured by Perkin-Elmer in USA), and the PCR amplification was purified with a QIAquick-spin PCR purification column (manufactured by QIAGEN Inc. in U.S.A.).

In addition, the RNA containing 2′-F-substituted pyrimidine was synthesized and purified through in vitro transcription with the use of a DuraScribe® T7 Transcription Kit (Lucigen, USA). Specifically, 200 pmoles of double-stranded DNA PCR amplification, 40 mM of tris-Cl (pH 8.0), 12 mM of MgCl₂, 5 mM of DTT, 1 mM of spermidine, 0.002% of Triton X-100, 4% of PEG 8000, 5 U of T7 RNA polymerase, and nucleotides including 1 mM of ATP, 1 mM of GTP, 3 mM of 2′F-CTP, and 3 mM of 2′F-UTP, were reacted at 37° C. for 6 to 12 hours, and the resultant product was purified with a Bio-Spin 6 chromatography column (Bio-Rad Laboratories, U.S.A.).

### Example 1-3

### Preparation of Conjugate (PLK1 siRNA AS/Peptide) of PLK1 siRNA Antisense Strand and Peptide

In this example, the antisense strand of the PLK1 siRNA, which is an oligonucleotide, and each of the five peptides in Table 1 were conjugated to prepare the PLK1 siRNA AS/peptide, which is a peptide conjugate with the antisense strand of the PLK1 siRNA. In this process, an antibody-oligonucleotide all-in-one conjugation kit (Solulink Inc. in USA) including an S-4FB linker represented by Formula 1 or an S-SS-4FB linker represented by Formula 2, which is a reagent that converts an amino functional group to a highly reactive aldehyde functional group, was used.

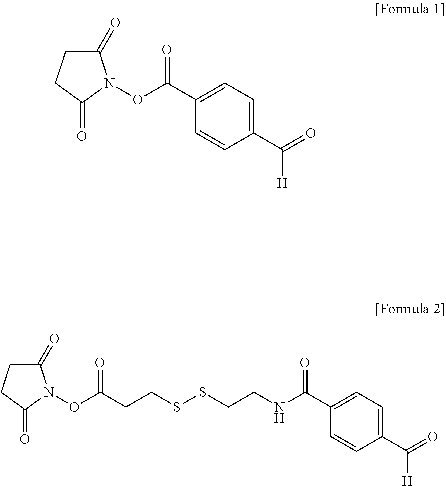 [SMILES: O=Cc1ccc(C(=O)ON2C(=O)CCC2=O)cc1 ; O=Cc1ccc(C(=O)NCCSSCCC(=O)ON2C(=O)CCC2=O)cc1]

First, with the use of the single-stranded nucleic acid DNA (“PLK1 siRNA AS”) of SEQ ID NO: 12 indicating the antisense strand of PLK1 siRNA as a template, an PLK1 siRNA antisense strand (PLK1 siRNA AS) in which an amino group is attached to the 5′ end and which contains a 2′-F-substituted pyrimidine was custom-made (Bioneer, Korea).

Next, 100 μl of an oligo resuspension solution of the antibody oligonucleotide all-in-one conjugation kit (Solulink, USA) was added to the freeze-dried product of the PLK1 siRNA AS to prepare a 0.5 OD260/μl PLK1 siRNA AS solution. The PLK1 siRNA AS solution was desalted with an oligo desalting spin column. To this desalted PLK1_siRNA_AS solution, a solution of S-4FB dissolved in DMF was added to the desalted PLK1_siRNA_AS solution to prepare the 0.5 OD260/μl PLK1 siRNA AS solution and, a reaction was performed in the resulting solution at room temperature for 2 hours so that the S-4FB linker having an aldehyde functional group linked was induced to be linked to the amino functional group. When this reaction was completed, the desalting process was performed as described above, and the resultant was collected.

Next, the peptides in Table 1 (Biocompare, USA) were concentrated to a concentration of 7.1 mg/ml using a 100 mM potassium phosphate buffer solution (pH 5.49). In addition, the PLK1 siRNA AS modified with the S-4FB linker was dissolved in a 50% dimethyl sulfoxide (DMSO) solvent to a concentration of 2.5 mg/ml. Next, the two solutions were mixed so that the molar ratio of the peptide and the PLK1_siRNA AS modified with the S-4FB linker was 1:9.

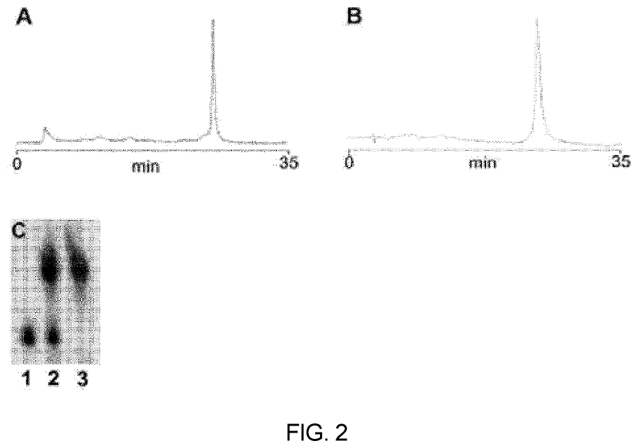

NaCNBH3 (Sigma, USA) was added to the mixed reaction solution to become a final concentration of 20 mM and was then reacted with slow stirring at 4° C. for 12 hours. A Sephadex G-25 column (GE Healthcare, USA) or a Resourcesphenyl column (GE Healthcare, USA) was used to separate the remaining peptide and S-4FB linker that were unreacted and the modified PLK1 siRNA AS. As a result, a conjugate in which PLK1 siRNA AS was selectively bound to the amino terminus of a peptide such as LPE3-1 was prepared. The PLK1 siRNA AS/peptide conjugate thus prepared was analyzed with HPLC and was then identified through non-denaturing (15%) polyacrylamide gel electrophoresis (PAGE) and SYBR gold staining (see FIG. 2). For reference, FIG. 2 shows the result of the case where LPE3-1 was used as a peptide. FIG. 2A is the HPLC analysis result of the reaction mixture of the PLK1 siRNA AS and the LPE3-1 peptide, and FIG. 2B is the HPLC analysis of the purified conjugate. FIG. 2C shows the PAGE images of (1) the PLK1 siRNA AS, (2) the reaction mixture of the PLK1 siRNA AS and the LPE3-1 peptide, and (3) the purified conjugate.

### Example 1-4

### Preparation of Drug Delivery System

The drug delivery system of the present invention was prepared by forming a double strand between the HER2 Ap/PLK1 siRNA SS conjugate and the PLK1 siRNA AS/peptide conjugate.

To form a double-stranded conjugate between the HER2 Ap/PLK1 siRNA SS conjugate and the PLK1 siRNA AS/peptide conjugate, 50 μM of the HER2 Ap/PLK1 siRNA SS conjugate and 50 μM PLK1 of the PLK1 siRNA AS/peptide conjugate were thermally denatured with an annealing buffer solution (10 mM of Tris-HCl at pH 7.4, 50 mM of NaCl, and 1 mM of ethylene diamine tetraacetic acid) at 95° C. for 3 minutes and were then slowly cooled to 20° C. to induce conjugation between the HER2 Ap/PLK1 siRNA SS conjugate and the PLK1 siRNA AS/peptide conjugate.

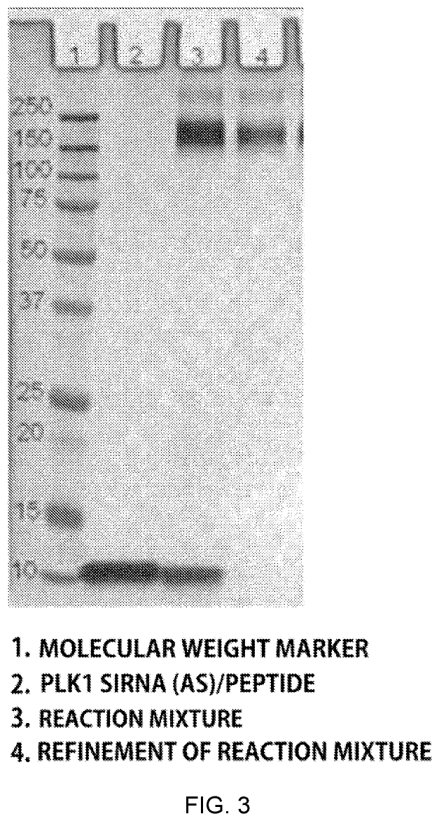

The conjugate resulting from the conjugation between the HER2 Ap/PLK1 siRNA SS conjugate and the PLK1 siRNA AS/peptide conjugate was desalted with an oligo desalting spin column as described above, was purified by Millipore centrifugation with a 0.22 μm sterile filtration membrane and was identified through non-denaturing (15%) polyacrylamide gel iontophoresis and ethidium bromide staining (see FIG. 3).

In FIG. 3, the first column is a molecular weight marker, the second column is a PLK1 siRNA AS/peptide conjugate sample, the third column is a crude reaction mixture of the HER2 Ap/PLK1 siRNA SS conjugate and the PLK1 siRNA AS/peptide conjugate and is a sample containing the PLK1 siRNA AS/peptide conjugate, and the fourth column is the analysis result of the sample from which the PLK1 siRNA AS/peptide conjugate is removed, in which the sample is obtained by purifying the reaction mixture of the HER2 Ap/PLK1 siRNA SS conjugate and the PLK1 siRNA AS/peptide conjugate.

### Example 2

### Selection of Peptides with Excellent Drug Release Properties

### Example 2-1

### Measurement of Apoptosis

In Example 1, the apoptosis effect of the drug delivery systems prepared by using the respective five peptides of Table 1 was investigated using a propidium iodide (PI) staining method, a phospho-H2AX analysis method, and an Annexin V FITC Apoptosis detection kit (BD corporation, USA).

BT-474 cells overexpressing the HER2 gene and MDA-MB-231 cells not expressing the HER2 gene were inoculated in a concentration of 10⁵ cells per well in a 12-well plate and then cultured, followed by stabilization in a cell incubator at 37° C. for 24 hours. Next, to induce apoptosis, each of the five drug delivery systems prepared in Example 1 was added, and the cells were cultured. The cells were treated with PI and Hoechst, respectively, at a final concentration of 1 μg/mL, at the time of 40 minutes before the end of the 72-hour culture. The sample plates were analyzed in real time with a High-Content Screening (HCS) system (ThermoFisher Scientific Inc., USA). To investigate phospho-H2AX, which is an early indicator of apoptosis, the treated cells were fixed with 2% paraformaldehyde and Hoechst dye for 30 minutes, then permeabilized with Triton X-100, and reacted at room temperature for 1 hour after bovine serum albumin (Sigma-Aldrich, USA) and mouse anti-human phospho-H2AX (Abcom, USA; 1:100 dilution) were added thereto. Next, rabbit anti-mouse Alexa Fluor 488 antibody (Invitrogen, USA; 1:100 dilution) was added thereto. After each step, the cells were gently washed with PBS. Finally, the sample plate was analyzed and the images were analyzed with an HCS system.

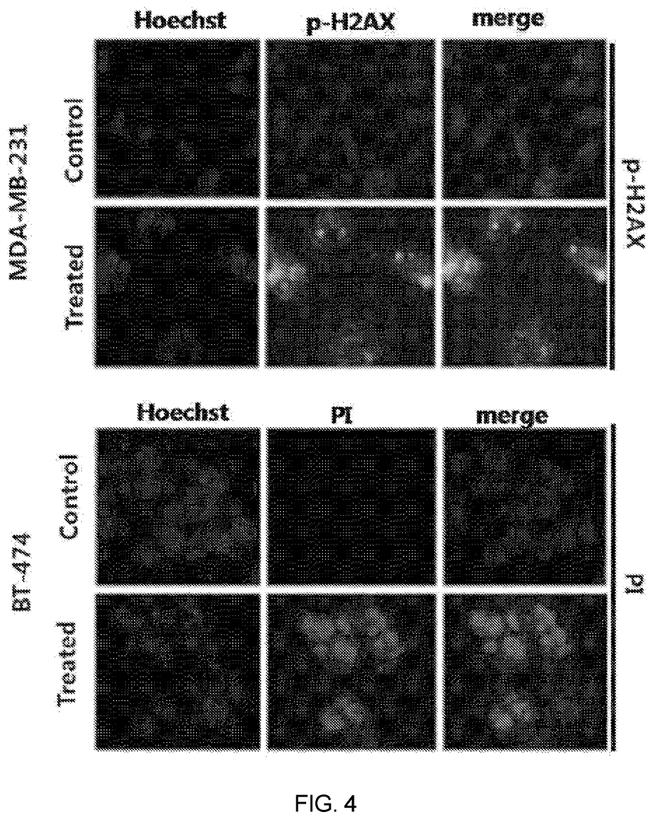

Normal living cells are negative for phospho-H2AX and PI, but cells undergoing early apoptosis are positive for phospho-H2AX and negative for PI. Cells undergoing late apoptosis are positive for PI (see FIG. 4).

FIG. 4 shows the results of treatment of the HER2-targeting drug delivery system prepared as described above in a cell culture solution of BT-474 cells overexpressing HER2 and a cell culture solution of MDA-MB-231 cells not expressing HER2. When treating the HER2-targeting drug delivery system, BT-474 which is a cell line overexpressing HER2 is positive for PI, thereby indicating the BT-474 cells are in the late apoptosis stage. On the other hand, MDA-MB-231 which is a cell line that does not express HER2 is positive for phospho-H2AX, indicating that the MDA-MA-231 cells are in the early apoptosis stage. These results show that the HER2-targeting drug delivery system of the present invention is more actively introduced into the HER2-positive cell line “BT-474” than into the HER2-negative cell line “MDA-MB-231”, thereby inducing active apoptosis in the BT-474 cells.

Meanwhile, for analysis using an Annexin V FITC apoptosis detection kit, BT-474 cells overexpressing the HER2 gene and MDA-MB-231 cells not expressing the HER2 gene were prepared and put in a 96-well culture vessel and then stabilized in a cell incubator at 37° C. for 24 hours. Next, 50 nM of each of the five drug delivery systems prepared in Example 1 was treated for 72 hours to induce apoptosis. The treated cells were washed twice with cold PBS, and then suspended in a 1X binding buffer at a concentration of 1×10⁶ cells/ml. Next, 100 μl of the solution (1×10⁵ cells/ml) was added to a 5 ml culture tube, and 5 pl of FITC Annexin V and 5 μl of propidium iodide (PI) were added. The solution was gently vortexed and incubated for 15 minutes in the dark at room temperature (25° C.). Next, 400 μl of the 1X Binding Buffer was added to each tube and analyzed by flow cytometry within 1 hour. The double fluorescence signal of the cells was analyzed using a microcapillary flow cytometer (BD corporation, USA).

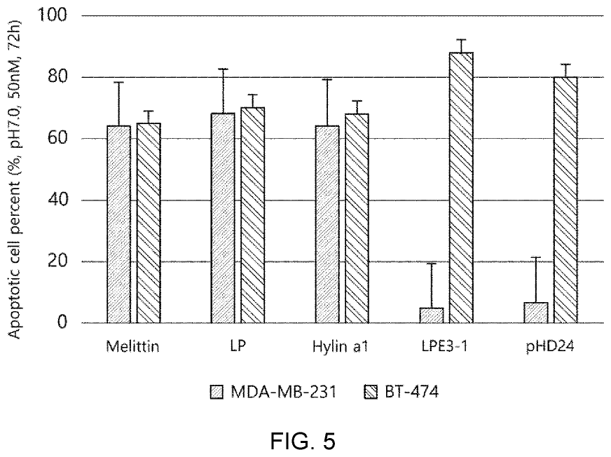

In this way, the antitumor ability (apoptotic effect) of each of the five types of drug delivery systems was investigated at a concentration of 50 nM and a pH of 7.0, and the results are shown in FIG. 5. Referring to FIG. 5, among the five drug delivery systems, the drug delivery systems using melittin, LP, or Hylin a1, each of which is a pH-independent cell-penetrating peptide, induced an apoptotic effect on both of the MDA-MB-231 cells that do not express the HER2 gene and the BT-474 cells that overexpress the HER2 gene. The drug delivery systems using LPE3-1 or pHD24, each of which is a pH-dependent cell-penetrating peptide, did not induce an apoptotic effect on the MDA-MB-231 cells not expressing the HER2 gene but induced an apoptotic effect on the BT-474 cells overexpressing the HER2 gene.

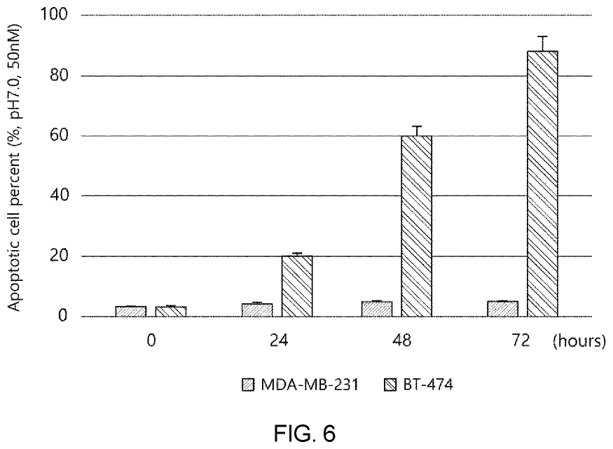

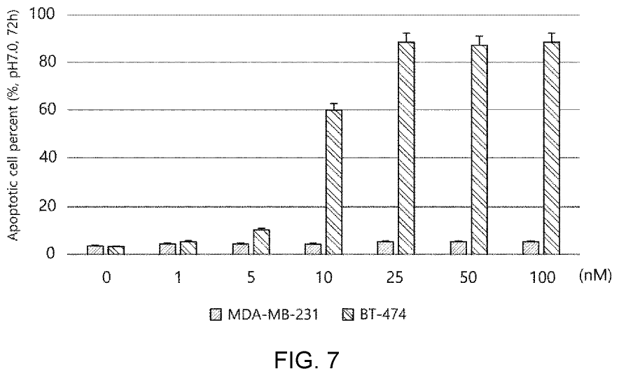

FIGS. 6 and 7 show the apoptosis induction effect according to the treatment time and treatment concentration of each of the drug delivery systems respectively using HER2 Ap, PLK1 siRNA, and LPE3-1 at a pH of 7.0. The treatment time and concentration of the drug delivery systems had little effect on the apoptosis of the MDA-MB-231 cells not expressing the HER2 gene, but it was observed that the effect on the apoptosis of the BT-474 cells overexpressing the HER2 gene was increased with increasing treatment time and treatment concentration.

The above results show that when a domain such as an aptamer specific to a target of a specific cell is used in combination with a peptide such as pHD24 and LPE3-1 having pH-dependent cell-penetrating activity, the drug delivery systems specifically act on specific cells that express target molecules at a pH of about 7.0 which is the environment of living organisms, thereby exhibiting the effect of reducing drug side effects.

### Example 2-2

### Measurement of Changes in Mitochondrial Membrane Potential

A flow cytometry mitochondrial membrane potential detection kit (BD Biosciences, USA) was used to detect changes in mitochondrial membrane potential. A cell sample was prepared in the same manner as in the apoptosis assay described above. 1 ml of a cell solution (1×10⁶ cells/ml) was transferred to a 15 ml culture tube and centrifuged at 800 rpm for 5 minutes, the supernatant was removed, 0.5 ml of a JC-1 solution was added to the precipitate, and the cells in the precipitate were cultured at room temperature (25° C.) in a dark place for 15 minutes. The precipitate was washed with 1 ml of an 1x assay buffer at 800 rpm for 5 minutes and was then centrifuged. After repeating the process described above twice, 0.5 ml of a 1x assay buffer was added to suspend the precipitate. Finally, the double fluorescence signal of the 0.5 mL solution was analyzed using a micro capillary flow cytometer (BD, USA). In addition, the pH of a cell culture medium was adjusted with an acid or alkali solution if necessary.

The changes in the cell mitochondrial membrane potential of cells were measured with the flow cytometry mitochondrial membrane potential detection kit. The measurement results are recorded as apoptosis, which is often associated with depolarization of ΔΨ, so the number of cells with reduced JC-1 fluorescence in the FL-2 channel increases. That is, apoptotic populations often exhibit a lower red fluorescence signal intensity (FL-2 axis) than negative control groups. In some apoptotic systems, changes in the level of green fluorescence measured in FL-1 were also observed.

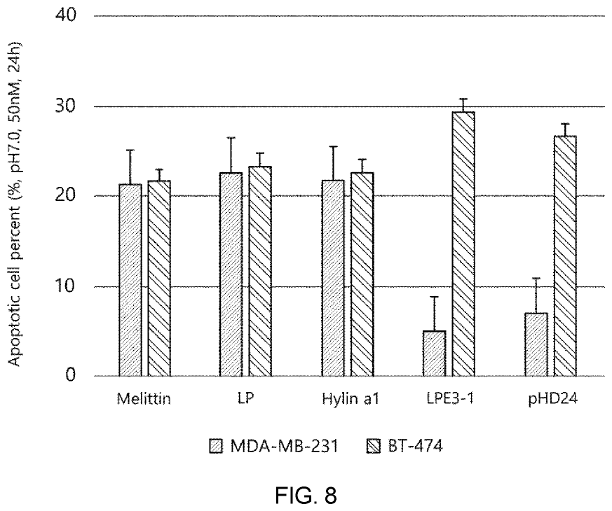

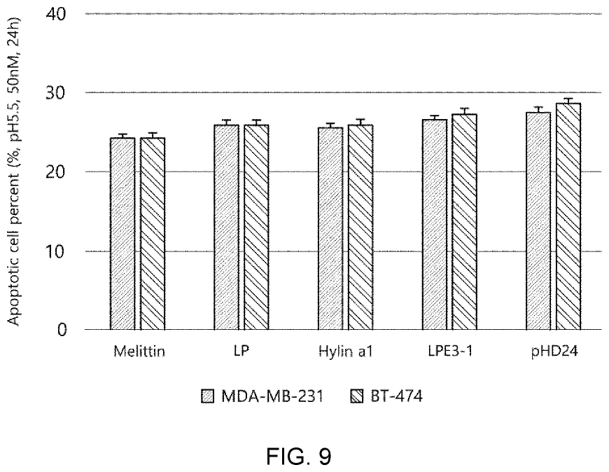

To confirm pH-dependent release, after treatment with the five types of drug delivery systems, the degree of apoptosis obtained by measuring the change in mitochondrial membrane potential of the BT-474 cells overexpressing the HER2 gene and the MDA-MB-231 cells not expressing the HER2 gene was investigated. The results are shown in FIGS. 8 and 9.

The MDA-MB-231 cells not expressing the HER2 gene were treated with each of the drug delivery systems that respectively contain melittin, LP, Hylin a1, LPE3-1, and pHD24 at a treatment concentration of 50 nM and a pH 7.0 for 24 hours, and the changes in mitochondrial membrane potential in the MDA-MB-231 cells not expressing HER2 gene were measured to observe the apoptosis effect. According to the results, the drug delivery systems respectively using melittin, Hylin a1, and LP showed an effect of 22.0% on average, and the drug delivery systems respectively using LPE3-1 and pHD24 showed an effect of 6.0% on average (see FIG. 8). In BT-474 cells overexpressing the HER2 gene, the drug delivery systems respectively using melittin, Hylin a1, and LP showed an average effect of 23.0%, and the drug delivery systems respectively using LPE3-1 and pHD24 showed an average effect of 28.0% (see FIG. 8).

Under conditions of a treatment concentration of 50 nM and a pH 5.5, the apoptosis effect was observed on the basis of changes in the mitochondrial membrane potential of the MDA-MB-231 cells in which the HER2 gene was not expressed. The drug delivery systems respectively using Melittin, Hylin, a1 and LP exhibited an apoptosis effect of 25.0% on average, and the drug delivery systems respectively using LPE3-1 and pHD24 exhibited an apoptosis effect of 27.0%. On the other hand, for the BT-474 cells overexpressing the HER2 gene, the drug delivery systems respectively using Melittin, Hylin a1, and LP exhibited an average apoptosis effect of 25.0%, and the drug delivery systems respectively using LPE3-1 and pHD24 showed an average apoptosis effect of 28.0% (see FIG. 9).

It was found that the apoptosis analysis result obtained with the use of the flow cytometry mitochondrial membrane potential detection kit was similar to the analysis result obtained with the use of the Annexin V FITC apoptosis detection kit as in Example 2-1.

The results of the examples show that when a peptide having a pH-dependent cell-penetrating activity is used in combination with a domain that recognizes a target of a specific cell, it acts only on cells expressing the target, thereby reducing the side effects caused by acting on cells that do not express the target.

### Example 3

### Preparation of Drug Delivery System Containing Paclitaxel and the Like

### Example 3-1

### Preparation of Test Group Drug Delivery System

Drug delivery systems prepared in the present example are (1) HER2 Ap/PLK1 siRNA/LPE3-1, (2) HER2 Ab/PLK1 siRNA/LPE3-1, (3) HER2 Ap/PAX/LPE3-1, and (4) HER2 Ab/PAX/LPE3-1.

Here, HER2 Ab refers to an antibody specific to HER2, and PAX refers to paclitaxel. The paclitaxel is a diterpenoid anticancer drug that is widely used as an anticancer drug for breast cancer and uterine cancer.

### 3.1.1

Preparation of HER2 Ap/PLK1 siRNA/LPE3-1

HER2 Ap/PLK1 siRNA/LPE3-1 drug delivery system is a drug delivery system composed of: an RNA aptamer containing 2′-F-substituted pyrimidine, having the ability to specifically bind to HER2, and serving as a cell targeting domain (CTD); PLK1 siRNA as a drug; and LPE3-1 peptide as a drug release domain (DRD). The drug delivery system was prepared in the same manner as in Example 1.

### 3.1.2 Preparation of HER2 Ab/PLK1 siRNA/LPE3-1

HER2 Ab/PLK1 siRNA/LPE3-1 is a drug delivery system composed of a HER2-specific antibody (ABCAM, USA) as a CTD, PLK1 siRNA as a drug, and LPE3-1 peptide as a DRD.

First, in order to manufacture a HER2 Ab and PLK1 siRNA SS conjugate, a PLK1 siRNA SS having the sequence of SEQ ID NO: 15, including a 2′-F-substituted pyrimidine and an introduced amino group at the 5′ end, was obtained from BioSynhesis (USA) by custom order.

(SEQ ID NO: 15)
UGA AGA AGA UCA CCC UCC UUA UU

Next, an oligo resuspension solution included in the Antibody-Oligonucleotide All-in-One Conjugation Kit (Solulink, USA) was added to lyophilized PLK1 siRNA SS to prepare a 0.5 OD260/μl solution. A desalting process was performed on the prepared PLK1 siRNA SS solution using a spin column (red cap) for oligo desalting. A solution of S-SS-4FB dissolved in DMF was added to the desalted PLK1 siRNA SS solution to prepare a 0.5 OD260/μl oligo solution and, a reaction was performed in the solution at room temperature for 2 hours so that an S-SS-4FB linker was bound to an amino functional group of the PLK1 siRNA SS. When this modification reaction was completed, a desalting process was performed, followed by a collection process.

Next, HER2 Ab was thickened to a concentration of 7.1 mg/ml with 100 mM of a potassium phosphate buffer (pH 5.49). In addition, the PLK1_siRNA_SS modified with the S-SS-4FB linker was dissolved in a solvent of 50% dimethyl sulfoxide (DMSO) to a concentration of 2.5 mg/m I.

Next, the two solutions were mixed so that the molar ratio of the HER2 Ab and the PLK1 siRNA SS modified with the S-SS-4FB linker was 1:9.

NaCNBH3 (Sigma, USA) was added to the reaction solution to be 20 mM and was then reacted with slow stirring at 4° C. for 12 hours. A Sephadex G-25 column (GE Healthcare, USA) or a Resourcesphenyl column (GE Healthcare, USA) was used to separate the HER2_Ab and S-SS-4FB linker that were not reacted and the modified PLK1 siRNA SS. As the final outcome, a conjugate in which the PLK1 siRNA SS was selectively bound to the amino terminus of the HER2 Ab was prepared.

A PLK1 siRNA AS/LPE3-1 conjugate was prepared in the same manner as in Example 1.

To form a double-stranded conjugate by binding the HER2 Ab/PLK1 siRNA SS conjugate and the PLK1 siRNA AS/LPE3-1 conjugate, 50 μM of the HER2 Ab/PLK1 siRNA SS conjugate and 50 μM the PLK1 siRNA AS/LPE3-1 conjugate were thermally denatured with an annealing buffer solution (10 mM of Tris-HCl at pH 7.4, 50 mM of NaCl, and 1 mM of ethylene diamine tetraacetic acid) at 95° C. for 3 minutes and were then slowly cooled to 20° C. to induce conjugation between the HER2 Ab/PLK1 siRNA_SS conjugate and the PLK1 siRNA AS/LPE3-1 conjugate.

The resulting double-stranded conjugate was desalted, purified by Millipore centrifugation with a 0.22 μm sterile filtration membrane, and identified through non-denaturing (15%) polyacrylamide gel iontophoresis and ethidium bromide staining (the results are not shown in the drawings). The amount of the double-stranded conjugate was measured with a spectrophotometer on the basis of the calculated molar absorption coefficient at λ=260 nm, and the purity of the drug delivery system having the HER2 Ab/PLK1 siRNA/LPE3-1 structure was analyzed by RP-HPLC.

### 3.1.3 Preparation of HER2 Ap/PAX/LPE3-1

A HR2 Ap/PAX/LPE3-1 drug delivery system is a drug delivery system composed of: an RNA aptamer (HER2 Ap) that is a cell-penetrating domain (CTD), has the ability to specifically bind to HER2, and contains a 2′-F-substituted pyrimidine; paclitaxel (PAX)(Sigma-Aldrich Inc., St Louis, USA); and an LPE3-1 peptide that is a drug release domain (DRD).

In this example, HER2 Ap and LPE3-1 peptide were first conjugated, and PAX was then bound thereto.

\textcircled{1} Preparation of HER2 Ap/LPE3-1 Peptide Conjugate

First, HER2 Ap was prepared in the same manner as in Example 1 as the RNA of SEQ ID NO: 16 including a spacer (underlined base sequence) and a 2′-F-substituted pyrimidine.

AGC CGC GAG GGG AGG GAA GGG UAG GGC GCG GCU-UUUU
(nucleotide sequence 16)

Next, the HER2 Ap/LPE3-1 peptide conjugate was prepared by the same method used to prepare the PLK1 siRNA AS/LPE3-1 peptide conjugate as in Example 1, except that HER2 Ap was used instead of PLK1 siRNA AS.

Next, in order to introduce a thiol functional group reactive with maleimide introduced into the PAX below into the HER2 Ap/LPE3-1 peptide conjugate, first, 2 mg of SPDP (Pierce Biotechnology, USA) was dissolved in 320 μL of DMSO to prepare a 20 mM SPDP reagent solution. 25 μL of the 20 mM SPDP solution was added to 2 to 5 mg of Ap-P dissolved in 1.0 mL of PBS-EDTA and was reacted at room temperature for 30 minutes. The desalting column was equilibrated with PBS-EDTA, and the buffer solution was exchanged to remove the reaction by-products and the excessive unreacted SPDP reagent.

23 mg of DTT was dissolved in PBS-EDTA to make a 150 mM DTT solution. A DTT solution was added to an SPDP-modified protein (to be a final concentration of 50 mM DTT) in a ratio of 0.5 mL DTT solution per mL SPDP-modified protein, followed by reaction for 30 minutes. The desalting column was equilibrated with PBS-EDTA and the protein was desalted to remove the DTT.

\textcircled{2} Synthesis of PAX with Maleimide Incorporated

For conjugation of the HER2 Ap/LPE3-1 peptide conjugate and the PAX, a thiol functional group and a reactive maleimide functional group were introduced into the PAX using 4-maleimidobutyric acid serving as a linker.

PAX 1g (1.17 mmol, 1 eq), 4-maleimidobutyric acid 210 mg (1.17 mmol, 1 eq), dimethylaminopyridine (DMAP) 140 mg (2.34 mmol, 2 eq), dicyclohexylcarbodiimide (DCC) 480 mg (1.17 mmol, 1 eq) were put in a 100-ml round flask, and 50 ml of methylene chloride was added thereto, followed by stirring for reaction at room temperature.

The progress of the reaction was observed using a thin layer chromatography (TLC) method. When the reaction was completed, 50 ml of distilled water (DW) was added thereto and shaken. (Rf Value=0.43, hexane:ethyl acetate=1:1)

The organic solvent layers were collected and water was removed with the use of magnesium sulfide, and then the organic solvent layers were separated by silica gel column chromatography. The material obtained through the hexane:ethyl acetate=1:1 silica gel column chromatography was concentrated to obtain 620 mg of a maleimide-introduced PAX compound.

\textcircled{3} Preparation of Conjugate of Maleimide-Introduced PAX and Thiol Functional Group-Introduced HER2 Ap/LPE3-1 Peptide Conjugate

100 mg (98 mol, 1eq) of the synthesized maleimide-introduced PAX and 100 mg (48 mol, 2eq) of the synthesized thiol-introduced HER2 Ap/LPE3-1 peptide conjugate were each dissolved in 1 mL of DMSO, and then the two solutions were mixed. Next, 2 to 3 drops of diisopropyl ethyl amine (DIPEA) were added thereto, and the solution mixture was reacted in a vortex for 5 minutes. The completion of the reaction was confirmed with Elman's reagent. When the yellow color disappeared, cooled diethyl ether was added to the obtained mixture, and then the mixture was centrifugated to obtained a precipitated compound. After the compound was purified by Prep-HPLC, the molecular weight thereof was measured by LC/MS, and the compound was frozen to produce a powder.

### 3.1.4 Preparation of HER2 Ab/PAX/LPE3-1

A HER2 Ab/PAX/LPE3-1 drug delivery system is a drug delivery system composed of a HER2-specific antibody (HER2 Ab) as a cell-penetrating domain (CTD), paclitaxel (PAX) as a drug, and LPE3-1 peptide as a drug release domain (DRD).

In this example, HER2 Ab and LPE3-1 peptide were first conjugated, and PAX was then bound thereto.

For conjugation of HER2 Ab and LPE3-1 peptide, 10 mg/ml HER2 Ab and 10 mg/ml LPE3-1 peptide were each dissolved in 0.1M N-morpholino ethanesulfonic acid (MES) buffer solution (pH 5). In addition, 1-Ethyl-3-(3-Dimethylaminopropyl) carbodiimide (EDAC) was dissolved in distilled water to a concentration of 10 mg/ml. The LPE3-1 peptide solution and the HER2 Ab solution were mixed, and the EDAC solution was added thereto. Then, the reaction was carried out at room temperature for 2 to 3 hours to induce conjugation of HER2 Ab and LPE3-1 peptide. The resulting product was desalted (using Cellu Sep dialysis) and stored after the buffer thereof was exchanged with an appropriate buffer (typically, PBS # UP30715).

The thiol functionalization of the HER2 Ab/LPE3-1 conjugate was performed using the SPDP reagent and the like in the same manner as in Example 3.1.3.

Next, the maleimide-introduced PAX prepared in Example 3.1.3 and the thiol-introduced HER2 Ab/LPE3-1 conjugate were reacted as in Example 3.1.3 to finally obtain a HER2_Ab/PAX/LPE3-1 drug delivery system.

### Example 3-2

### Preparation of Control Group Drug Delivery System

Drug delivery systems prepared in this example were prepared as a control group with respect to the test group prepared in Example 3-1. Specifically, (1) HER2 Ap/LPE3-1, (2) HER2 Ab/LPE3 -1, (3) HER2 Ap/PLK1 siRNA, (4) HER2 Ap/PAX, (5) HER2 Ab/PLK1_siRNA, (6) HER2 Ab/PAX, (7) LPE3-1/PLK1 siRNA, and (8) LPE3-1/PAX were prepared.

(1) Preparation of HER2 Ap/LPE3-1

This was prepared in the same manner as in Example 3.1.3.

(2) Preparation of HER2 Ab/LPE3-1

This was prepared in the same manner as in Example 3.1.4.

(3) Preparation of HER2 Ap/PLK1 siRNA

HER2 Ap/PLK1 siRNA is a double-stranded conjugate of HER2 Ap/PLK1 siRNA SS and PLK1 siRNA AS. It was prepared in the same manner as in Example 1, except that PLK1 siRNA AS was used instead of siRNA AS/peptide conjugate in the double-stranded conjugate formation reaction.

(4) Preparation of HER2 Ap/PAX

A SH-HER2 Ap containing a thiol functional group and a 2′-F-substituted pyrimidine for HER2 was manufactured by BioSynhesis (USA) by custom order.

The preparation of PAX having a maleimide functional group introduced was carried out in the same manner as in Example 3.1.3.

Conjugation of the PAX into which the maleimide functional group was introduced and the SH-HER2_Ap was performed in the same manner as in Example 3.1.3.

(5) Preparation of HER2 Ab/PLK1 siRNA

HER2 Ab/PLK1 siRNA is a double-stranded conjugate of a HER2 Ab/PLK1 siRNA SS conjugate and PLK1 siRNA AS. It was prepared in the same manner as in Example 3.1.2, except that PLK1 siRNA AS was used instead of the PLK1 SiRNA AS/LPE3-1 conjugate in the double-stranded conjugate formation reaction.

(6) Preparation of HER2 Ab/PAX

The introduction of the thiol functional group into the HER2 Ab was performed in the same manner as in Example 3.1.3, except that HER2_Ab was used instead of the HER2 Ap/LPE3-1 conjugate.

The conjugation of the PAX into which the maleimide functional group was introduced and the HER2 Ab was performed in the same manner as in Example 3.1.3.

(7) Preparation of LPE3-1/PLK1 siRNA

LPE3-1/PLK1 siRNA is a double-stranded conjugate of an LPE3-1/PLK2_siRNA_SS conjugate and PLK1 siRNA_AS. First, the preparation of the LPE3-1/PLK2 siRNA_SS conjugate was performed using an LPE3-1 peptide that was purchased and a PLK1 siRNA that was custom-made by BioSynhesis (USA) in which an amino group is present at the 5′ end. The preparation was performed by the same method of preparing the HER2-Ap/LPE3-1 conjugate as in Example 3.1.3. Next, a double-stranded conjugate was formed using the PLK1 siRNA AS in the same manner as in Example 1.

(8) Preparation of LPE3-1/PAX

The introduction of the thiol functional group into LPE3-1 was performed in the same manner as in Example 3.1.3, except that LPE3-1 peptide was used instead of the HER2 Ap/LPE3-1 conjugate. Next, the PAX into which a maleimide functional group was introduced and the LPE3-1 peptide into which the thiol functional group was introduced were conjugated in the same manner as in Example 3.1.3.

### <Example 4

### Anticancer Activity of Drug Delivery System Containing Paclitaxel

As a cell line for confirming anticancer activity, BT-474 and MDA-MB-231 cell lines were used as in Example 2.

The drug delivery systems used to confirm the anticancer activity were four the test group drug delivery systems prepared in Example 3-1, including (1) HER2 Ap/PLK1 siRNA/LPE3-1, (2) HER2 Ab/PLK1 siRNA/LPE3-1, (3) HER2 Ap/PAX/LPE3-1, and (4) HER2 Ab/PAX/LPE3-1, and the eight control group drug delivery systems prepared in Example 3-2, including (1) HER2 Ap/LPE3-1, (2) HER2 Ab/LPE3-1, (3) HER2 Ap/PLK1 siRNA, (4) HER2 Ap/PAX, (5) HER2 Ab/PLK1 siRNA, (6) HER2 Ab/PAX, (7) LPE3-1/PLK1 siRNA, and (8) LPE3-1/PAX.

The anticancer activity was confirmed by measuring the degree of cell death in cancer cell lines according to the treatment concentration of the drug delivery system, using the Annexin V FITC Apoptosis detection kit (BD, USA).

The BT-474 cell line and the MDA-MB-231 cell line were put in a 96-well culture vessel and then stabilized in advance in a cell incubator at 37° C. for 24 hours. Thereafter, the cell lines were treated with each of the test group drug delivery systems and each of the control drug delivery systems for each concentration for 72 hours, and the degree of apoptosis was measured in the same manner as in Example 2.

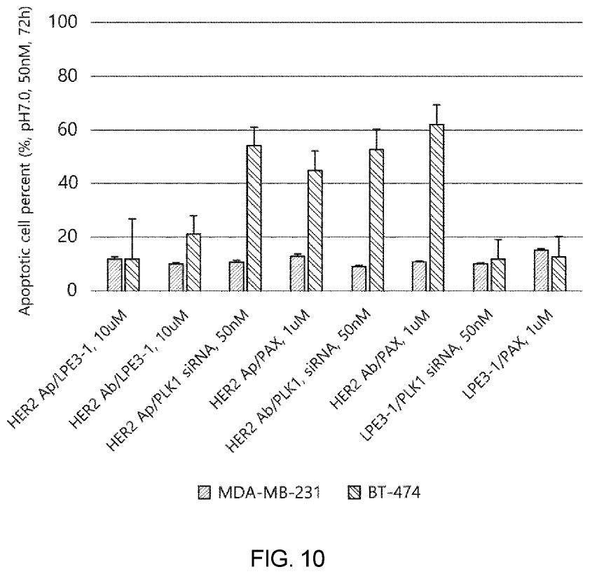

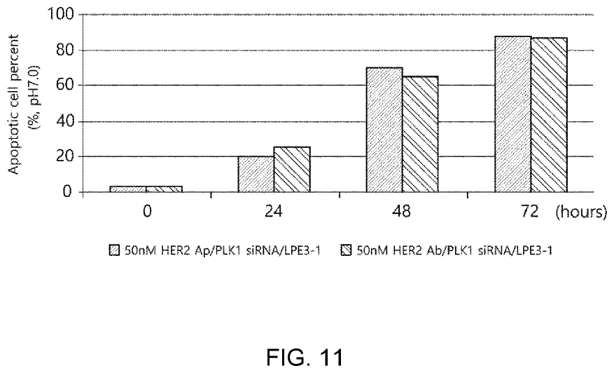

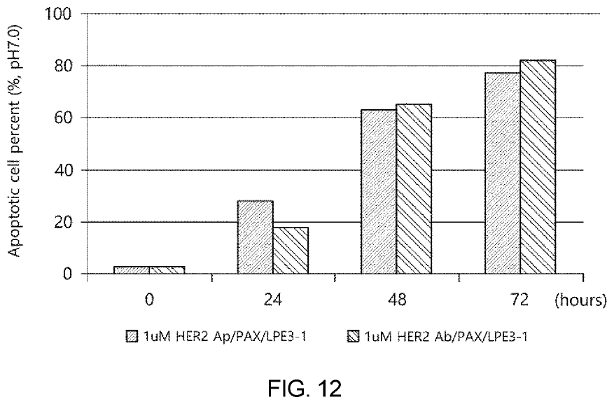

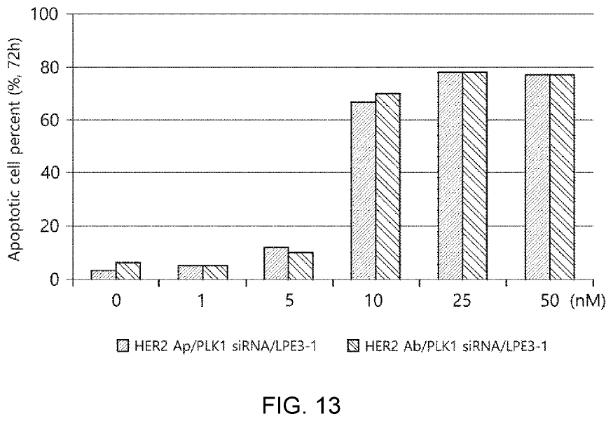

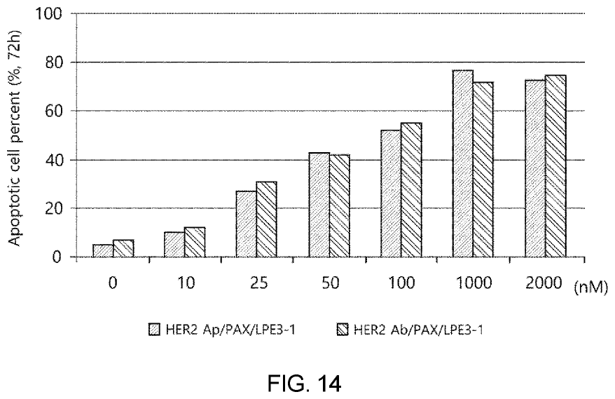

The degree of apoptosis of the BT-474 cell line and the MDA-MB-231 cell line, when the cell lines were treated by each of the control group drug delivery systems during 72-hour culture at a treatment concentration of 50 nM and a pH of 7.0, is expressed, in FIG. 10, as a percentage compared to the untreated group. The degree of apoptosis of the BT-474 cell line overexpressing the HER2 gene for each treatment time, when the cell line was treated by each of the test group drug delivery systems at a treatment concentration of 50 nM or 1 μM and a pH of 7.0, is expressed, in FIGS. 11 and 12, as a percentage compared to the untreated group. The degree of apoptosis of the BT-474 cell line overexpressing the HER2 gene for each treatment concentration of each of the test group drug delivery systems during 72-hour culture at a pH of 7, is expressed, in FIGS. 13 and 14, as a percentage compared to the untreated group.

Referring to FIG. 10, among the control group drug delivery systems, the HER2 Ap/LPE3-1 and the HER2 Ab/LPE3-1 had no anticancer effect on both the BT-474 cell line overexpressing the HER2 gene and the MDA-MB-231 cell line not expressing the HER2 gene. The HER2 Ap/PLK1 siRNA, HER2 Ap/PAX, HER2 Ab/PLK1 siRNA, and HER2 Ab/PAX had anticancer effects only on the BT-474 cell line overexpressing the HER2 gene. In addition, the LPE3-1/PLK1 siRNA and LPE3-1/PAX had no anticancer effect on both the BT-474 cell line overexpressing the HER2 gene and the MDA-MB-231 cell line not expressing the HER2 gene. The results of the apoptosis of the control group drug delivery systems suggest that the HER2-specific aptamer or antibody binds to the BT-474 cell line overexpressing the HER2 gene and internalizes into the cell as endosomes, the endosomes are oxidized, and a portion of the drug delivery system is released into the cytoplasm. Due to the mechanism, 45% to 62% of the cells were killed.

In addition, as confirmed from FIGS. 11 to 14, all of the four drug delivery systems in the test group had no anticancer effect on the MDA-MB-231 cell line in which the HER2 gene was not expressed (data not shown). On the other hand, the investigation of the anticancer activity of the four drug delivery systems in the test group with respect to the BT-474 cell line showed that the drug delivery systems “HER2 Ap/PLK1 siRNA/LPE3-1” and “HER2 Ab/PLK1 siRNA/LPE3-1” that include PLK1 siRNA as a drug had an increasing cancer cell killing effect according to an increasing culture time (see FIG. 11). In addition, EC50 (concentration corresponding to 50% apoptosis) appeared in a zone where 100 nM or more of the drug delivery system was administered, and a maximum of 78% apoptosis was observed (see FIG. 13). The drug delivery systems “HER2 Ap/PAX/LPE3-1” and “HER2 Ab/PAX/LPE3-1” that include PAX as a drug exhibited an increasing cancer cell killing effect in proportion to the culture time (see FIG. 12), and EC50 appeared in a zone where 100 nM or more of the drug delivery system was administered, and a maximum of 77% apoptosis and a maximum of 75% apoptosis were respectively observed for the respective drug delivery systems (see FIG. 14).

The results of the anticancer activity confirmation experiment presented in the above examples showed that the test group drug delivery systems having a cell targeting domain had a large anticancer effect on BT-474 cells overexpressing the HER2 gene and little anticancer effect on the MDA-MB-231 cells not expressing the HER2 gene. This means that a small amount of anticancer agent (PLK1 siRNA or paclitaxel) can have a large anticancer effect on cells with a specific target molecule, thereby reducing the side effects of anticancer treatment and increasing the effect of the anticancer drug.

In addition, the results show that the administration of the test group drug delivery systems consisting of a cell targeting domain (CTD), a drug, and a drug release domain (DRD) can show drug efficacy even at a small concentration compared to the administration of the control group drug delivery systems.

The present invention has been described and illustrated with reference to some specific embodiments thereof, and those skilled in the art will appreciate that various adaptations, changes, modifications, substitutions, deletions, or additions of methods or protocols may be made without departing from the spirit and scope of the invention. Accordingly, the present invention is defined only by the scope of the appended claims, and the scope of the claims should be construed as broadly as reasonable.

1. A drug delivery system comprising: (a) a cell targeting domain that is a region that specifically binds to a target molecule of a target cell; and (b) a pH-dependent cell-penetrating peptide bound to the cell targeting domain.

2. The drug delivery system according to claim 1, wherein the target molecule is an antigen or receptor present on the surface of the target cell.

3. The drug delivery system according to claim 1, wherein the target cell is a cancer cell, an abnormal cell, or a normal cell.

4. The drug delivery system according to claim 1, wherein the cell targeting domain is an antibody, an antibody fragment, an aptamer, a hormone, a cytokine, a chemokine, a ligand, a peptide as a partial region of a cytokine, or a peptide as a partial region of a ligand, each of which is capable of binding to the target molecule.

5. The drug delivery system according to claim 1, wherein the pH-dependent cell-penetrating peptide is a GALA peptide, a pHD15 peptide that is a variant of the MelP5 peptide, a pHD24 peptide that is a variant of the MelP5 peptide, a pHD108 peptide that is a variant of the MelP5 peptide, an LPE3-1 peptide that is a variant of an LP peptide, an LPH4 peptide that is a variant of the LP peptide, or an ATRAM peptide.

6. The drug delivery system apparatus according to claim 1, wherein the cell targeting domain is bound to the pH-dependent cell-penetrating peptide (i) directly covalently, (ii) non-covalently, (iii) via a linker, or (iv) via a biocompatible polymer.

7. The drug delivery system according to claim 1, wherein the cell targeting domain and the pH-dependent cell-penetrating peptide bind to each other via a linker, and
the linker has, as a functional group, isothiocyanate, isocyanates, acyl azide, NHS ester, sulfonyl chloride, aldehyde, glyoxal, epoxide, oxirane, carbonate, aryl halide, imidoester, carbodiimide, anhydride, fluorophenyl ester, hydroxymethyl phosphine, maleimide, haloacetyl, pyridyldisulfide, thiosulfonate, or vinylsulfone.

8. The drug delivery system according to claim 1, wherein the cell targeting domain and the pH-dependent cell-penetrating peptide bind to each other via a linker, and
the linker is a cleavable linker or a non-cleavable linker.

9. The drug delivery system apparatus according to claim 8, wherein the cleavable linker is a linker cleavable by a protease, a linker cleavable under acid or base conditions, or a linker cleavable under reducing or oxidizing conditions, and
the non-cleavable linker is a linker including maleimidomethyl cyclohexane-1-carboxylate (MCC) and maleimidocaproyl (MC).

10. The drug delivery system according to claim 1, wherein the cell targeting domain and the pH-dependent cell-penetrating peptide bind to each other via a linker, and
the linker having two or more functional groups.

11. The drug delivery system according to claim 10, wherein the linker having two or more functional groups is a homobifunctional linker, a heterobifunctional linker, or a resin-type linker.

12. The drug delivery system according to claim 1, wherein the cell targeting domain and the pH-dependent cell-penetrating peptide bind to each other via a biocompatible polymer.

13. The drug delivery system according to claim 1, wherein the biocompatible polymer is a synthetic polymer or a natural polymer.

14. A drug and drug delivery system conjugate comprising a drug bound to the drug delivery system of claim 1.

15. The drug and drug delivery system conjugate according to claim 14, wherein the drug is bound to the cell targeting domain or the pH-dependent cell-penetrating peptide of the drug delivery system, (i) directly covalently, (ii) non-covalently, (iii) via a linker.

16. The drug delivery system according to claim 14, wherein the drug is bound to the drug delivery system (i) in sequential order of the drug, the cell targeting domain, and the pH-dependent cell-penetrating peptide, (ii) in sequential order of the cell targeting domain, the drug, and the pH-dependent cell-penetrating peptide, or (iii) in sequential order of the cell targeting domain, the pH-dependent cell-penetrating peptide, and drug.

17. The drug delivery system according to claim 14, wherein the drug is bound to the drug delivery system via a biocompatible polymer.

18. The drug and drug delivery system conjugate according to claim 14, wherein the drug is a drug that moves to the cytoplasm of a cell and exhibits a therapeutic effect therein.

19. The drug and drug delivery system conjugate according to claim 14, wherein the drug is a low molecular compound drug, gene, plasmid DNA, antisense oligonucleotide, siRNA, peptide, ribozyme, viral particle, immunomodulator, protein, or contrast agent.

20. The drug and drug delivery system conjugate according to claim 14, wherein the drug is a cytotoxic anticancer agent, and the cytotoxic anticancer agent is antimetabolites, microtubulin targeting agents (tubulin polymerase inhibitor and tubulin depolymerisation), alkylating agents, antimitotic agents, DNA cleavage agents, DNA cross-linker agents, DNA intercalator agents, or DNA topoisomerase inhibitors.


SEQUENCE LISTING (16 sequences)

SEQ ID NO: 1 - protein, 26 aa, Artificial Sequence
  Penetrating Peptide
GWWLALAEAEAEALALASWIKRKRQQ

SEQ ID NO: 2 - protein, 26 aa, Artificial Sequence
  Penetrating Peptide
GWWLALALALALALALASWIHHHHQQ

SEQ ID NO: 3 - protein, 26 aa, Artificial Sequence
  Penetrating Peptide
GIGEVLHELADDLPDLQEWIHAAQQL

SEQ ID NO: 4 - protein, 26 aa, Artificial Sequence
  Penetrating Peptide
GIGDVLHELAADLPELQEWIHAAQQL

SEQ ID NO: 5 - protein, 26 aa, Artificial Sequence
  Penetrating Peptide
GIGEVLHELAEGLPELQEWIHAAQQL

SEQ ID NO: 6 - protein, 34 aa, Artificial Sequence
  Penetrating Peptide
GLAGLAGLLGLEGLLGLPLGLLEGLWLGLELEGN

SEQ ID NO: 7 - protein, 26 aa, Artificial Sequence
  Penetrating Peptide
GIGAVLKVLTTGLPALISWIKRKRQQ

SEQ ID NO: 8 - protein, 26 aa, Artificial Sequence
  Penetrating Peptide
GWWLALALALALALALASWIKRKRQQ

SEQ ID NO: 9 - protein, 18 aa, Artificial Sequence
  Penetrating Peptide
IFGAILPLALGALKNLIK

SEQ ID NO: 10 - DNA, 60 bp, Artificial Sequence
  Aptamer-spacer-PLK1 siRNA sense strand
agccgcgaggggagggaagggtagggcgcggcttttttgaagaagatcaccctccttatt

SEQ ID NO: 11 - DNA, 23 bp, Artificial Sequence
  PLK1 siRNA sense strand
tgaagaagatcaccctccttatt

SEQ ID NO: 12 - DNA, 22 bp, Artificial Sequence
  PLK1 siRNA anti-sense strand
taaggagggtgatctttcttca

SEQ ID NO: 13 - DNA, 41 bp, Artificial Sequence
  primer
taatacgactcactatagggagaagccgcgaggggagggaa

SEQ ID NO: 14 - DNA, 18 bp, Artificial Sequence
  primer
aataaggagggtgatctt

SEQ ID NO: 15 - RNA, 23 nt, Artificial Sequence
  PLK1 siRNA sense strand
ugaagaagaucacccuccuuauu

SEQ ID NO: 16 - RNA, 37 nt, Artificial Sequence
  Aptamer
agccgcgaggggagggaaggguagggcgcggcuuuuu